(19) United States
(12) Patent Application Publication
(10) Pub. No.: US 2026/0007648 A1
(43) Pub. Date: Jan. 8, 2026

(54) COMPOUND WITH STING DEGRADATION ACTIVITY, AND COMPOSITION AND APPLICATION THEREOF

(71) Applicant: ZHONGSHAN OPHTHALMIC CENTER, SUN YAT-SEN UNIVERSITY, Guangzhou (CN)

(72) Inventors: Lili GONG, Guangzhou (CN); Xialin LIU, Guangzhou (CN); Xingfei ZHU, Guangzhou (CN); Wei LIU, Guangzhou (CN)

(21) Appl. No.: 19/266,129

(22) Filed: Jul. 10, 2025

(30) Foreign Application Priority Data
Jul. 10, 2024 (CN) 202410919874.0

(51) Int. Cl. A61K 31/454; A61P 27/02; A61P 29/00; A61P 35/00; C07D 401/04

(52) U.S. Cl. CPC A61K 31/454; A61P 27/02; A61P 29/00; A61P 35/00; C07D 401/04

(57) ABSTRACT

A compound with a STING degradation activity, which is represented by formula (I): X-Y-Z where X is

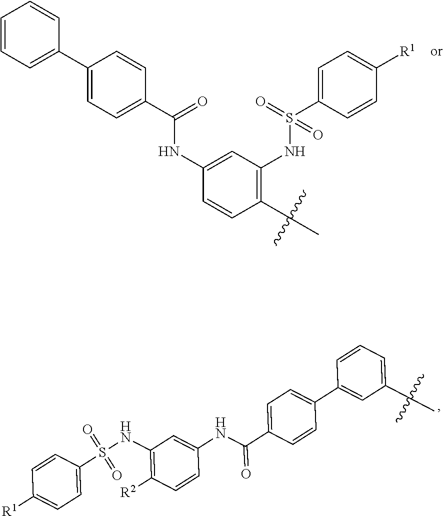

Y is a linker, and Z is a group capable of binding to an E3 ubiquitin ligase. A pharmaceutical composition including the compound of formula (I) and a pharmaceutically acceptable carrier is provided. This application also provides a method for treating a STING function-related disease with such compound, where the disease includes an autoimmune disease and an inflammatory disease.

### CROSS-REFERENCE TO RELATED APPLICATIONS

This application claims the benefit of priority from Chinese Patent Application No. 202410919874.0, filed on Jul. 10, 2024. The content of the aforementioned application, including any intervening amendments thereto, is incorporated herein by reference in its entirety.

### TECHNICAL FIELD

This application relates to pharmaceutical synthesis, and more particularly to a compound with a STING degradation activity, and a composition and application thereof.

### BACKGROUND

Stimulator of Interferon Genes (STING) is a transmembrane protein located on the endoplasmic reticulum of cells. It has been found in various tissues, and plays a key role in the innate immune system of cells. Specifically, cGAS binds to double-stranded DNA, generating the 2′-3′-cyclic GMP-AMP (cGAMP), which is a ligand for STING. After binding to cGAMP, STING translocation from ER to golgi, where it is activated and subsequently recruits the downstream kinase TBK1. Phosphorylation/activation of TBK1 then activates transcription factor IRF3 and/or NFKB, and thus induces production of interferons and cytokines, thereby participating in the antiviral immune response and autoimmune regulation.

Due to the crucial role of the STING in the cellular innate immune response, its abnormal activation is associated with various diseases, especially those related to the immune system. For instance, the abnormal STING signal may cause the immune system to mistakenly attack the host's own tissues, thereby triggering the autoimmune diseases; and the excessive activation of the STING will promote the production of inflammatory cytokines, which may be related to chronic inflammatory diseases. In view of this, small molecules targeting STING have emerged as a new therapeutic strategy for treating these diseases.

Ubiquitin, a 76-amino acid protein found in eukaryotic cells, is characterized by a highly conserved sequence. It mainly plays a role in labelling target proteins, and the labeled target protein can be recognized and degraded by the proteasome (referred to as ubiquitin-proteasome pathway). The E3 ubiquitin ligase is directly attached to the target protein, and thus greatly determines the recognition specificity. The degradation process involving the ubiquitin-proteasome system mainly includes:
1) ubiquitin activation: the carboxyl residue of ubiquitin is bound to the sulfhydryl group of the ubiquitin-activating enzyme E1;
2) ubiquitin conjugation: the activated ubiquitin is transferred from E1 to the ubiquitin-conjugating enzyme E2 through a transesterification process;
3) binding of ubiquitin complex to target protein by E3: the ubiquitin-E2 complex is attached to the target protein under the action of the ubiquitin-protein ligase E3; and
4) proteasomal degradation: the ubiquitinated target protein is recognized by the proteasome, and then hydrolyzed into peptide fragments with a length of 7-8 amino acid residues.

Proteolysis targeting chimeric molecules (PROTACs) are bifunctional molecules that can simultaneously bind to the E3 ubiquitin ligase and the target protein. Under the action of PROTACs, the target protein that is originally unable to bind to E3 can be ubiquitinated, and then selectively degraded through the ubiquitin-proteasome system, ultimately achieving the regulation of the intracellular level of the target protein. The current E3 ubiquitin ligases mainly include cereblon (CRBN), von Hippel-Lindau (VHL), mouse double minute 2 (MDM2), and cellular inhibitor of apoptosis protein-1 (cIAP1). PROTACs have three essential components, including a warhead, an E3 ligase ligand, and a linker conjugating the two moieties. The selection and optimization of the warhead, and the composition, length, and attachment site of the linker will significantly determine the constructed PROTAC.

Compound SP23 has been reported as a STING-targeted PROTAC degrader exhibiting an excellent degradation efficiency. However, it struggles with a relatively high cytotoxicity, which greatly limits its clinical application.

### SUMMARY

A first object of the present disclosure is to provide a compound having a degradation activity against the STING, as well as significantly-reduced cytotoxicity (demonstrated by in-vivo (mouse model) and in-vitro (THP1, ARPE, SH-SY5Y cell lines) experiments).

A second object of the present disclosure is to provide a pharmaceutical composition including the above compound.

A third object of the present disclosure is to provide an application of the above compound in the preparation of a drug for preventing and/or treating diseases associated with STING activity, or in the preparation of a drug for preventing and/or treating inflammatory diseases and/or autoimmune diseases.

A fourth object of the present disclosure is to provide a method for preventing and/or treating diseases associated with the STING function.

A fifth object of the present disclosure is to provide a method for treating ocular diseases.

The technical solutions adopted by the present disclosure will be specifically described as follows.

In a first aspect, the present disclosure provides a compound of formula (I), or a deuterated compound, a stereoisomer, a tautomer, a polymorph, a solvate, a N-oxide, an isotope-labeled compound, a metabolite, a prodrug or a pharmaceutically acceptable salt thereof:

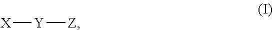

wherein X is

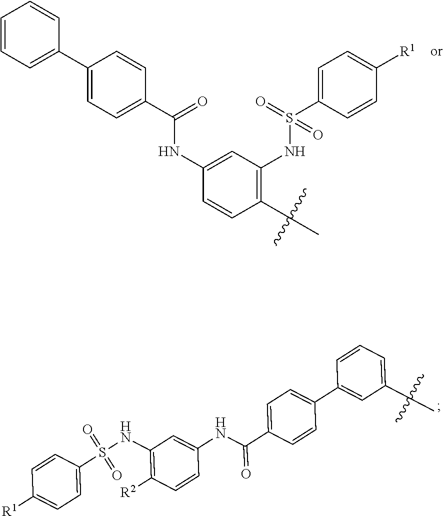

R¹ is selected from the group consisting of hydrogen, halogen, cyano, nitro, hydroxyl, —C₁₋₆ alkyl, —C₂₋₆ alkenyl, —C₂₋₆ alkynyl, halogen-substituted —C₁₋₆ alkyl, halogen-substituted —C₂₋₆ alkenyl, halogen-substituted —C₂₋₆ alkynyl, —O(C₁₋₆ alkyl), —NH₂, —NH(C₁₋₆ alkyl), and —N(C₁₋₆ alkyl)(C₁₋₆ alkyl);
R² is selected from the group consisting of hydrogen, halogen, cyano, nitro, hydroxyl, —C₁₋₆ alkyl, —C₂₋₆ alkenyl, —C₂₋₆ alkynyl, halogen-substituted —C₁₋₆ alkyl, halogen-substituted —C₂₋₆ alkenyl, halogen-substituted —C₂₋₆ alkynyl, —O(C₁₋₆ alkyl), —NH₂, —NH(C₁₋₆ alkyl), and —N(C₁₋₆ alkyl)(C₁₋₆ alkyl);
Y is a linking group;
Z is a group capable of binding to an E3 ubiquitin ligase.

In some embodiments, R¹ is selected from the group consisting of hydrogen, fluorine, chlorine, bromine, iodine, cyano, hydroxyl, methyl, ethyl, n-propyl, isopropyl, vinyl, ethynyl, monofluoromethyl, difluoromethyl, trifluoromethyl, methoxy, ethoxy, —NH₂, —NH(methyl) and —N(methyl)(methyl); preferably, R¹ is selected from the group consisting of fluorine; and
R² is selected from hydrogen, fluorine, chlorine, bromine, iodine, cyano, hydroxyl, methyl, ethyl, n-propyl, isopropyl, vinyl, ethynyl, monofluoromethyl, difluoromethyl, trifluoromethyl, methoxy, ethoxy, —NH₂, —NH(methyl), —N(methyl)(methyl) and preferably, R² is selected from the group consisting of hydroxyl.

In some embodiments, X is selected from the group consisting of

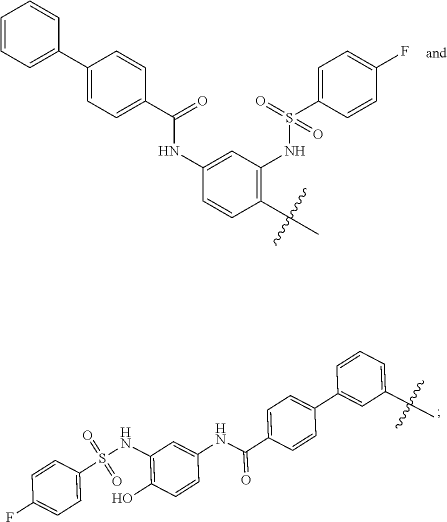 [SMILES: *c1ccc(NC(=O)c2ccc(-c3ccccc3)cc2)cc1NS(=O)(=O)c1ccc(F)cc1 ; *c1cccc(-c2ccc(C(=O)Nc3ccc(O)c(NS(=O)(=O)c4ccc(F)cc4)c3)cc2)c1]

In some embodiments, Y is selected from -(L^{Y})_{q}-;
q is an integer selected from 1-30; and each L^{Y} is independently selected from C(R)₂, C(O), O, S, S(O), S(O)₂, NR, —CR═CR—, —C≡C—, 3-10-membered cycloalkyl, 3-10-membered heterocycloalky, 6-10-membered aryl, 5-10-membered heteroary, 5-12-membered spiro group, 5-12-membered spiroheterocycly, 5-12-membered bridged ring group, and 5-12-membered bridged heterocyclyl;
wherein cycloalkyl, heterocycloalkyl, aryl, heteroaryl, spiro group, spiroheterocyclyl, bridged ring group and bridged heterocyclyl are unsubstituted or substituted by one, two or three R^{YL} groups;
wherein each R^{YL} group is independently selected from the group consisting of hydrogen, halogen, cyano, nitro, —C₁₋₆ alkyl, halogen-substituted —C₁₋₆ alkyl, —OR and —N(R)(R); and
each R is independently selected from the group consisting of hydrogen, halogen, —C₁₋₆ alkyl and halogen-substituted —C₁₋₆ alkyl.

In some embodiments, Y is

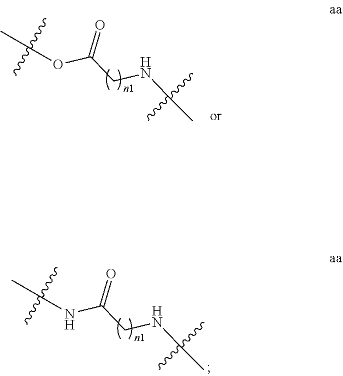

wherein aa indicates an end linked to X;
and n1 is an integer selected from 1-10; preferably, n1 is 0, 1, 2, 3, 4, 5, 6, 7 or 8.

In some embodiments, Y is selected from the group consisting of:

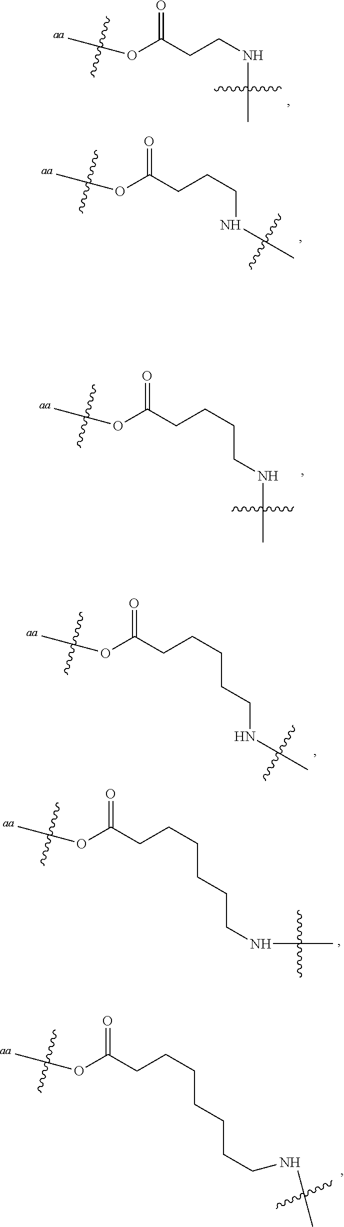

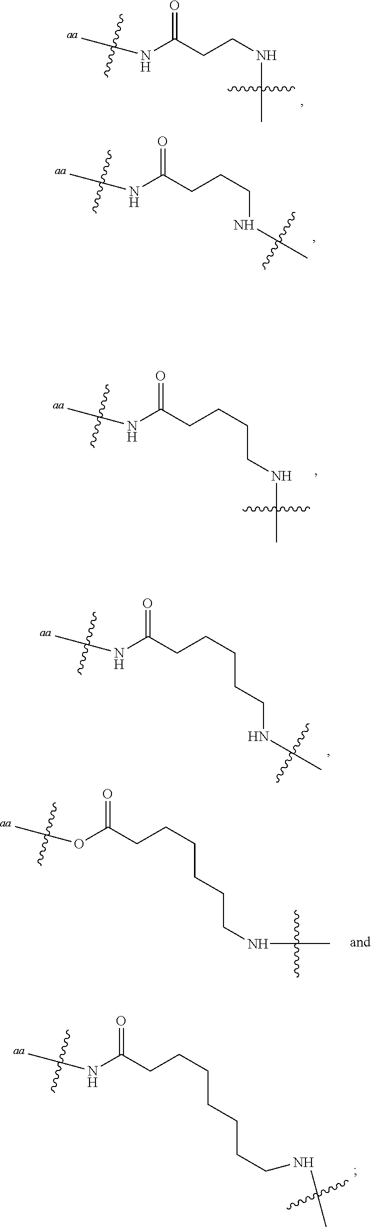

wherein aa terminal indicates an end linked to X.

In some embodiments, the E3 ubiquitin ligase is selected from the group consisting of Cereblon (CRBN) E3 ubiquitin ligase, von Hippel-Lindau (VHL) E3 ubiquitin ligase, X-linked inhibitor of apoptosis protein (XIAP), mouse double minute 2 homolog (MDM2) and cellular inhibitor of apoptosis protein 1 (cIAP-1). Preferably, the E3 ubiquitin ligase is selected from the group consisting of Cereblon (CRBN) E3 ubiquitin ligase;
ubiquitin ligases, also known as E3 ubiquitin ligases, are enzymes that link ubiquitin molecules to a lysine of a target protein;
cereblon (CRBN), a component of the E3 ubiquitin ligase complex, is the target of immunomodulatory drugs such as thalidomide;
von Hippel-Lindau or VHL is an E3 ligase;
X-linked inhibitor of apoptosis protein (XIAP) is a major member of the IAP family; phosphorylation of XIAP functions as a ubiquitin ligase;
MDM2 (Murine double minute 2) is one of the important members of RING (Really interesting new gene) type ubiquitin protein ligases; and
cellular inhibitor of apoptosis protein-1 (cIAP-1) is an inhibitor of apoptosis protein-1, which also has ubiquitin ligase activity.

In some embodiments, Z is selected from the group consisting of:

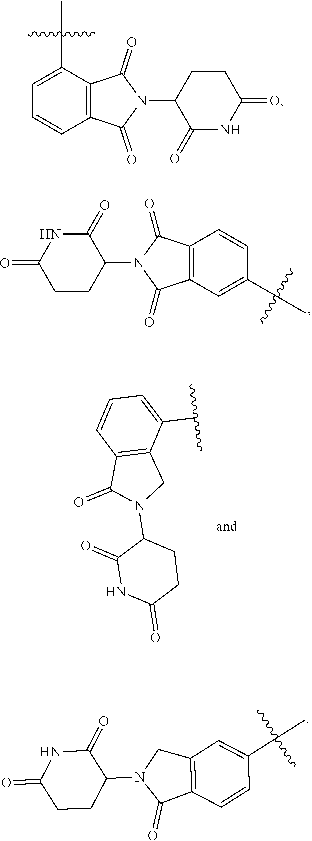 [SMILES: *c1cccc2c1C(=O)N(C1CCC(=O)NC1=O)C2=O ; *c1ccc2c(c1)C(=O)N(C1CCC(=O)NC1=O)C2=O ; *c1cccc2c1CN(C1CCC(=O)NC1=O)C2=O ; *c1ccc2c(c1)CN(C1CCC(=O)NC1=O)C2=O]

In some preferred embodiments, the compound is selected from the group consisting of:

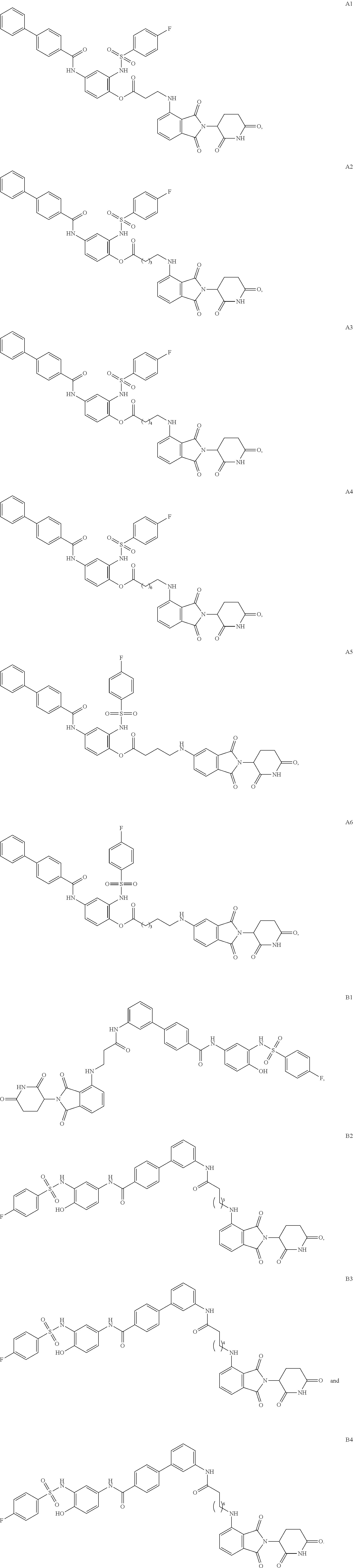

Generally, PROTAC includes have three essential components, including a warhead, an E3 ligase ligand, and a linker conjugating the two moieties. The selection and optimization of the warhead, and the composition, length, and attachment site of the linker will significantly determine the constructed PROTAC.

The compounds A1 (ZOC6), A2 (ZOC1), A3 (ZOC2), A4 (ZOC3), A5 (ZOC4), A6 (ZOC5), as well as B2 (ZOC8), B3 (ZOC9), and B4 (ZOC10) in the present disclosure all have a degrading effect on STING and significantly reduce cytotoxicity. The aforementioned compounds have the advantage of better security.

SP23 is the STING degradation inhibitor in the prior art. Although SP23 has a good degradation efficiency, its strong cytotoxicity greatly limits its clinical application. By optimizing the warhead molecule and linker design, a series of new compounds with simplified structure and smaller molecular weight (790-880 g/mol) are successfully produced.

The in-vitro cell assays results are summarized as follows:
the degradation efficiency of compound B2 (ZOC8) is similar to that of SP23 in ARPE cells, specifically, the maximum degradation rate of compound B2 is 72.2%, and the maximum degradation rate of SP23 is 71.8%;
in THP1 cells, the degradation efficiencies of compound A1 (ZOC6) and SP23 are similar; the maximum degradation rate of A1 is 76.6%, and the maximum degradation rate of SP23 is 87.8%; and
the cytotoxicity of A1 (ZOC6) and B2 (ZOC8) were 3-7 times lower than that of SP23 under the same treatment conditions.

In-vivo experiments show that the injection of SP23 causes a decline in visual function in mice, while the compound A1 (ZOC6) in the present disclosure has no adverse effect on the visual function.

In a second aspect, the present disclosure provides a pharmaceutical composition, including:
the above-described compound, or a deuterated compound, a stereoisomer, a tautomer, a polymorph, a solvate, a N-oxide, an isotope-labeled compound, a metabolite, a prodrug or a pharmaceutically acceptable salt thereof; and
a pharmaceutically acceptable carrier.

Commonly used pharmaceutically acceptable carriers or excipients include stabilizers, diluents, surfactants, lubricants, antioxidants, binders, colorants, fillers, emulsifiers, and the like.

The pharmaceutical composition may be formulated into tablets, capsules, emulsions, suspensions, dispersions, or solutions.

In a third aspect, the present disclosure provides the use of compounds as described in any one of the above claims, or their deuterated compound, stereoisomer, tautomer, polymorph, solvate, N-oxide, isotopically labeled compound, metabolite, prodrug, or a pharmaceutically acceptable salt thereof, or pharmaceutical composition in the preparation of a drug for preventing and/or treating diseases associated with the STING function.

In an embodiment, the diseases associated with the STING function include neurodegenerative diseases, inflammatory diseases, autoimmune diseases, metabolic diseases, fibrotic diseases, and other inflammatory/degenerative disorders.

In an embodiment, the STNG function-related disease is selected from the group consisting of STING-associated vasculopathy with onset in infancy (SAVI), Aicardi-Goutières syndrome (AGS), coatomer subunit α (COPA) syndrome caused by genetic variations in a subunit α of a coatomer protein complex, systemic lupus erythematosus (SLE), familial chilblain lupus (FCL), Parkinson's disease (PD), amyotrophic lateral sclerosis (ALS), Huntington's disease, non-alcoholic steatohepatitis (NASH), alcoholic liver disease, nerve injury, rheumatoid arthritis, renal fibrosis, systemic sclerosis, intervertebral disc degeneration, pulmonary fibrosis, aging, scleroderma, psoriasis, inflammatory bowel disease, autoimmune colitis, irritable bowel syndrome, ulcerative colitis, Crohn's disease, uveitis, mucositis, diabetes, and cardiovascular disease.

The present disclosure further provides the use of a compound as described above, or a deuterated compound, a stereoisomer, a tautomer, a polymorph, a solvate, a N-oxide, an isotope-labeled compound, a metabolite, a prodrug, or a pharmaceutically acceptable salt thereof, or a pharmaceutical composition, in the preparation of a drug for preventing and/or treating inflammatory diseases and/or autoimmune diseases.

In an embodiment, the inflammatory and autoimmune diseases include STING-associated vasculopathy with onset in infancy (SAVI), Aicardi-Goutières syndrome (AGS), COPA syndrome, systemic lupus erythematosus (SLE), familial chilblain lupus (FCL), rheumatoid arthritis, systemic sclerosis, psoriasis, inflammatory bowel disease, autoimmune colitis, irritable bowel syndrome, uveitis, and mucositis.

In a fourth aspect, the present disclosure provides a method for preventing and/or treating diseases associated with the STING function, including:
administering a therapeutically effective amount of the compounds according to the above claims, or a deuterated compound, a stereoisomer, a tautomer, a polymorph, a solvate, a N-oxide, an isotope-labeled compound, a metabolite, a prodrug, or a pharmaceutically acceptable salt thereof to the subject. In-vitro cell experiments and animal model studies demonstrated that the compounds of the present disclosure exhibited low toxicity profiles, making them suitable as prophylactic agents.

In an embodiment, an administration route of the compound is selected from the group consisting of oral administration, intravenous ingestion, intramuscular injection, and subcutaneous injection, transdermal administration, inhalation administration, sublingual administration, dermal administration, ocular administration, nasal administration, intra-articular administration and intrathecal administration.

The compounds or pharmaceutical compositions of the present disclosure may be administered alone or in combination with other therapeutic agents. Oral compositions may be in any orally acceptable dosage form, including but not limited to a tablet, a capsule, an emulsion, a suspension, a dispersion, and a solution.

In an embodiment, the topical administration for the skin, eyes, and nose is performed by ocular surface instillation or intravitreal injection.

In a fifth aspect, the present disclosure provides a method for treating an ocular disease, including administering a therapeutically effective amount of the pharmaceutical composition described above to the subjecting via intravitreal injection.

The indications of intravitreal injection mainly include vascular abnormalities (such as wet age-related macular degeneration), inflammatory reactions (such as uveitis edema), and infection (such as endophthalmitis), which can achieve rapid effect by targeted drug delivery and maintain a high therapeutic drug concentration at the lesion location.

The compounds and derivatives provided in the present disclosure may be named according to the IUPAC (International Union of Pure and Applied Chemistry) or CAS (Chemical Abstracts Service, Columbus, OH) nomenclature systems.

Definitions of terms for use in the present disclosure: unless otherwise stated, the initial definitions provided by the groups or terms herein apply to that group or term throughout the specification. For terms not specifically defined herein, they should be given the meaning that those skilled in the art can give them, according to the disclosure content and context.

“Substitution” refers to the replacement of a hydrogen atom in a molecule by another different atom or group; or the lone pair of electrons of an atom in a molecule is substituted by another atom or group.

“Unsubstituted or substituted” means that “substituted” may but must not occur, and this description includes circumstances in which it does or does not occur.

The minimum and maximum values of the carbon atom number in the hydrocarbon group are indicated by a prefix. For example, the prefix C_{a-b}-alkyl indicates any alkyl group containing “a” to “b” carbon atoms. Further, a C₁₋₆ alkyl group refers to an alkyl group containing one to six carbon atoms.

“Alkyl” refers to a saturated hydrocarbon chain having a specified number of atoms. Alkyl groups can be straight or branched. Representative branched alkyl groups have one, two, or three branched chains. The alkyl group may optionally be substituted by one or more substituents as defined herein. Alkyl groups include methyl, ethyl, propyl (n-propyl and isopropyl), butyl (n-butyl, isobutyl, and tert-butyl), pentyl (n-pentyl, isopentyl, and neopentyl), and hexyl. The alkyl group may also be part of other groups, such as —O(C₁₋₆ alkyl).

The term “cycloalkyl” in the present disclosure refers to divalent cyclic alkanes with multiple carbon atoms and no ring heteroatoms that are partially saturated or non-aromatic and having single or multiple rings (fused).

The term “cycloalkyl” includes cycloolefins, such as cyclohexalkenes. Examples of single-carbon cyclic groups include cyclopropane, cyclobutane, cyclohexane, cyclopentane, cyclooctane, cyclopentene and cyclohexolene. Examples of fused cycloalkyl system include dicyclohexane, dicyclopentane, dicyclooctane, etc.

The implementation of “cycloalkyl” as described in the present disclosure includes but is not limited to

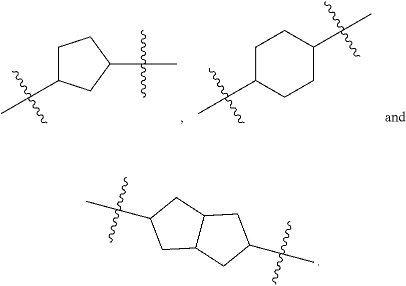 [SMILES: *C1CCC(*)C1 ; *C1CCC(*)CC1 ; *C1CC2CC(*)CC2C1]

The term “heterocycloalky” in the present disclosure refers to a saturated cyclic or non-aromatic partially saturated divalent cyclic group containing at least one heteroatom and having a single ring or multiple rings (fused), in which heteroatoms refer to nitrogen atoms, oxygen atoms, sulfur atoms, etc.

Examples of heterocycloalky in the monocyclic heterocycloalkane system include oxetane, azetidine, pyrrolidine, 2-oxo-pyrrolidine, tetrahydrofuran, tetrahydro-thiophene, pyrazolidine, imidazolidine, thiazolidine, piperidine, tetrahydropyran, tetrahydrothiopyran, piperazine, morpholine, thiomorpholine, 1,1-dioxo-thiomorpholine, azepane, diazepane, etc.

Examples of fused heterocycloalky systems include 8-aza-bicyclo[3.2.1]octane, quinuclidine, 8-oxa-3-aza-bicyclo[3.2.1]octane, 9-aza-bicyclo[3.3.1]nonane, etc.

The term “heterocycloalky” also includes partially saturated ring systems formed by the fusion of an aromatic ring and a non-aromatic ring, in which at least one heteroatom is present. The point of attachment may be located on a non-aromatic carbon atom, an aromatic carbon atom, or a heteroatom.

The term “spiro group” in the present disclosure refers to a saturated or non-aromatic partially saturated divalent cyclic group formed by the spiro group of two or more rings, which contain multiple carbon atoms and no ring heteroatoms.

The term “spiroheterocycly” in the present disclosure refers to a saturated or non-aromatic partially saturated divalent cyclic group formed by the spiro group of two or more rings, which contain at least one heteroatom.

The term “bridged ring group” in the present disclosure refers to a saturated or non-aromatic partially saturated divalent cyclic group formed by the bridging of multiple rings, which contain multiple carbon atoms and no ring heteroatoms.

The term “bridged heterocyclyl” in the present disclosure refers to a saturated or non-aromatic partially saturated divalent cyclic group formed by the bridging of multiple rings, which contain at least one heteroatom.

The term “unsaturated” in the present disclosure means that a group or molecule contains a carbon-carbon double bond, carbon-carbon triple bond, carbon-oxygen double bond, carbon-sulfur double bond, carbon-nitrogen triple bond, or the like.

The term “alkenyl” in the present disclosure refers to a straight or branched hydrocarbon group having at least one ethylenic unsaturation site (—C═C—). For example, a C_{a-b} alkenyl group refers to an alkenyl group having from a to b carbon atoms and is intended to include, such as vinyl, propenyl, isopropenyl, 1,3-butadienyl, and the like.

The term “alkynyl” in the present disclosure refers to a straight or branched monovalent hydrocarbon group containing at least one triple bond.

The term “alkynyl” is also intended to include those hydrocarbon groups having one triple bond and one double bond. For example, a C₂₋₆ alkynyl group is intended to include ethynyl, propynyl, and the like.

The term “aromatic ring” in the present disclosure refers to an aromatic hydrocarbon group containing multiple carbon atoms. Aryl groups are generally monocyclic, bicyclic, or tricyclic aryl groups with multiple carbon atoms. Additionally, the term “aryl” as used herein refers to an aromatic substituent that may be a single aromatic ring or multiple aromatic rings fused together. Non-limiting examples include phenyl, naphthyl, or tetrahydronaphthyl.

The term “aromatic heterocycle” in the present disclosure refers to an aromatic unsaturated ring containing at least one heteroatom; the heteroatom refers to a nitrogen atom, oxygen atom, sulfur atom, or the like. It is typically an aromatic monocyclic or bicyclic hydrocarbon containing multiple ring atoms, among which one or more ring atoms are heteroatoms selected from O, N, and S. Preferably, there are one to three heteroatoms.

Heterocyclic aryl groups refer to, for example, pyridyl, indolyl, quinoxalinyl, quinolinyl, isoquinolinyl, benzothienyl, benzofuranyl, benzothienyl, chromenyl, benzothiopyranyl, furyl, pyrrolyl, thiazolyl, oxazolyl, isoxazolyl, triazolyl, tetrazolyl, pyrazolyl, imidazolyl, thienyl, oxadiazolyl, benzimidazolyl, benzothiazolyl, and benzoxazolyl.

The term “halogen” in the present disclosure refers to fluorine, chlorine, bromine, or iodine.

The term “halogen-substituted alkyl” in the present disclosure refers to an alkyl group in which one or more hydrogen atoms are substituted by halogen atoms. For example, a halogen-substituted C₁₋₄ alkyl group refers to an alkyl group containing 1 to 4 carbon atoms with one or more hydrogen atoms substituted by halogen atoms. Further examples include monofluoromethyl, difluoromethyl, and trifluoromethyl.

The term “halogen-substituted alkenyl” in the present disclosure refers to an alkenyl group in which one or more hydrogen atoms are substituted by halogen atoms; for example, a halogen-substituted C₂₋₆ alkenyl group refers to an alkenyl group containing 2 to 6 carbon atoms with one or more hydrogen atoms substituted by halogen atoms. Further examples include monofluorovinyl, difluorovinyl, and trifluoropropenyl.

The term “halogen-substituted alkynyl” in the present disclosure refers to an alkynyl group in which one or more hydrogen atoms are substituted by halogen atoms; for example, a halogen-substituted C₂₋₆ alkynyl group refers to an alkynyl group containing 2 to 6 carbon atoms with one or more hydrogen atoms substituted by halogen atoms, such as monofluoroethynyl, difluoroethynyl, and trifluoropropynyl.

In the present disclosure, the terms such as “—OR” and “—N(R)₂” mean that the R group is connected to an oxygen atom or a nitrogen atom via a single bond.

The term “═O” in the present disclosure refers to an oxygen atom replacing two hydrogen atoms in the molecule through a double bond.

In groups such as “—C(O)R” and “—S(O)₂R” mentioned in the present disclosure, the oxygen atom is connected to a carbon atom or a sulfur atom via a double bond, and the R group is connected to the oxygen atom or sulfur atom via a single bond; for another example, “—S(O)(NH)R” means that the oxygen atom and nitrogen atom are connected to the sulfur atom via double bonds, and the R group is connected to the sulfur atom via a single bond.

The symbols “ [symbol] ” and “ [symbol] ” in the chemical group descriptions of the present disclosure are used to indicate the positions of chemical group substitutions.

The “deuterated compound” in the present disclosure refers to a compound in which one or more hydrogen atoms in the molecule or group are replaced by deuterium atoms, where the proportion of deuterium atoms is greater than the natural abundance of deuterium.

The term “pharmaceutically acceptable” means that a carrier, vehicle, diluent, excipient, and/or formed salt is generally chemically or physically compatible with other components constituting a pharmaceutical dosage form and physiologically compatible with the recipient.

The terms “salt” and “pharmaceutically acceptable salt” refer to acid and/or basic salts formed by the above-mentioned compounds or their stereoisomers with inorganic and/or organic acids and bases, including zwitterionic salts (inner salts) and quaternary ammonium salts such as alkylammonium salts. These salts can be obtained directly during the final isolation and purification of the compounds, or by mixing the above-mentioned compounds, or their stereoisomers, with an appropriate amount (e.g., an equivalent amount) of an acid or base. These salts may be collected by filtration after precipitation in solution, recovered after solvent evaporation, or prepared by freeze-drying after reaction in an aqueous medium.

The term “prevention” includes inhibiting and delaying the onset of a disease, and not only includes prevention before the development of the disease but also includes preventing the recurrence of the disease after treatment.

The term “treatment” means reversing, alleviating, or eliminating the progression of the disorder or condition to which the term applies, or one or more symptoms of such disorder or condition.

In some embodiments, one or more compounds of the present disclosure may be used in combination with each other. Alternatively, the compounds of the present disclosure may be used in combination with other active agents for the preparation of drugs or pharmaceutical compositions for regulating the cell function or treating diseases. If a group of compounds are used, these compounds may be administered to the subject simultaneously, separately, or orderly.

Obviously, based on the above content of the present disclosure in accordance with the ordinary knowledge and conventional means in the art, various modifications, substitutions, or changes can be made without departing from the above basic technical idea of the present disclosure.

The present disclosure will be described in detail below with reference to examples. It should be understood that these examples are merely illustrative, and are not intended to limit the present disclosure. All modifications, substitutions, or changes made without departing from the spirit of the disclosure shall fall within the scope of the disclosure defined by the appended claims.

The advantages of the present disclosure are as follows.

It has been confirmed by in-vitro cell experiments that the novel PROTAC small-molecule drugs developed in the present disclosure can effectively degrade the intracellular STING through the ubiquitin-proteasome pathway. Moreover, the specificity of such PROTAC small-molecule drugs has been verified in the STING-knockout mice.

In-vivo (mouse models) and in-vitro (THP1, ARPE, and SH-SY5Y cell lines) experiments have demonstrated that the PROTAC small molecule drugs of the present disclosure significantly reduced cytotoxicity, while maintaining a certain STING degradation efficiency. Their cytotoxicity is much lower than that of the STING degrader (SP23) in the prior art, showing better safety advantages. In particular, in animal model experiments involving intravitreal injection—a route of administration for ophthalmic drugs—it has been demonstrated that the PROTAC small-molecule drugs disclosed herein have no significant impact on retinal function, exhibiting excellent intraocular safety profiles.

The PROTAC small-molecule drugs of the present disclosure have been shown to exert a good inhibitory effect on systemic inflammatory responses in TREX1 knockout mice (the gold standard model for evaluating therapeutic strategies targeting the STING pathway). Further, these drugs effectively degraded STING, inhibited retinal inflammatory responses, and protected the retina in two retinal injury models with confirmed STING activation (light damage and retinal vasculitis induced by intravitreal injection of cGAMP).

The present disclosure provides a better option for the treatment of autoimmune diseases and inflammatory diseases associated with STING.

### BRIEF DESCRIPTION OF THE DRAWINGS

FIG. 1 is a ¹H NMR spectrum of Compound A1 (ZOC6) according to an embodiment of the present disclosure;

FIG. 2 is a ¹H NMR spectrum of Compound B2 (ZOC8) according to an embodiment of the present disclosure;

FIGS. 3A-B show Western Blot analysis results of the degradation effect of the compound according to an embodiment of the present disclosure on the STING in the adult retinal pigment epithelial cell line ARPE;

FIG. 4 shows micro-content protein electrophoresis analysis results of the degradation activity of the compound according to an embodiment of the present disclosure in the human leukemia monocytic cell line THP1;

FIGS. 5A-B show Western Blot analysis results of the degradation effect of the compound according to an embodiment of the present disclosure on the STING in the human neuroblastoma cells SH-SY5Y;

FIG. 6 shows the live-dead cell staining results of ARPE cells;

FIG. 7 shows the live-dead cell staining results of SH-SY5Y cells;

FIG. 8 shows statistical results of the live-dead cell staining of SH-SY5Y cells;

FIGS. 9A-C show representative mouse electroretinogram (ERG) waveforms of individual treatment groups in the mouse visual function ERG experiment, where A: DMSO treatment; B: ZOC6 treatment; and C: SP23 treatment;

FIGS. 10A-B are statistical graphs of the a-wave (10A) and b-wave (10B) amplitudes in the mouse ERG under different flash stimulation intensities;

FIGS. 11A-B show test procedures (A) and statistical analysis results (B) of the mouse visual acuity test;

FIG. 12A schematically shows the design of the mouse treatment experiment;

FIG. 12B shows mouse body weight changes over 14 days;

FIG. 13A shows gross images of heart, liver and kidney from mice 14 days after intravitreal injection of ZOC6 (100 μM);

FIGS. 13B-D show sizes of heart, liver and kidney from mice 14 days after intravitreal injection of ZOC6 (100 μM);

FIG. 14 is a hematoxylin and eosin (H&E)-stained image of paraffin sections of heart, liver and kidney from mice 14 days after intravitreal injection of ZOC6 (100 μM);

FIG. 15 is a H&E-stained paraffin section image of eyes from mouse 14 days after intravitreal injection of ZOC6;

FIGS. 16A-B shows the Western Blot results (A) and grayscale statistical analysis (B) to evaluate the effect of cGAMP transfection on tbk1 phosphorylation and inhibitory effect of zoc6;

FIGS. 17A-D show the immunofluorescence results;

FIGS. 18A-B show the enzyme-linked immunosorbent assay results;

FIGS. 19A-B shows the Western Blot results (A) and corresponding statistical analysis (B);

FIG. 20 shows the Western Blot results to assess the inhibitory effect of PROTAC (ZOC6) on STING downstream inflammatory responses;

FIG. 21 shows the statistical analysis of the Western Blot results in FIG. 20;

FIG. 22 shows H&E-stained paraffin section images of heart, liver and kidney of mice;

FIGS. 23A-C show inflammation statistical results of the H&E-stained images, where A: liver; B: heart; and C: kidney;

FIG. 24 shows the immunofluorescence staining results of ionized calcium-binding adapter molecule 1 (IBA1)-positive microglia in paraffin sections of mouse retina;

FIG. 25 shows the glial fibrillary acidic protein (GFAP) immunofluorescence staining results of paraffin sections of mouse retina;

FIG. 26 is a schematic diagram of the experimental design;

FIGS. 27A-B shows the Western Blot results (A) and corresponding statistical analysis (B) according to an embodiment of the present disclosure;

FIG. 28 is an optical coherence tomography (OCT) scan image;

FIG. 29 is a H&E-stained image of paraffin sections of mouse retina;

FIGS. 30A-B show the TUNEL staining (A) of mouse retina paraffin sections and corresponding statistical analysis (B);

FIGS. 31A-C are ERGs of mice from different treatment groups;

FIGS. 32A-B are statistical graphs of the a-wave and b-wave amplitudes of the mouse ERG under different flash stimulation intensities;

FIG. 33 shows the immunofluorescence staining results of protein kinase C (PKC)-α positive bipolar cells in paraffin sections of mouse retina;

FIG. 34 shows the immunofluorescence staining results of phosphodiesterase 6H (pde6h) positive photoreceptor cells in paraffin sections of mouse retina;

FIGS. 35A-B show the GFAP immunofluorescence staining results (A) in mouse retina and corresponding statistical analysis (B);

FIGS. 36A-B show the Western Blot results (A) of GFAP in mouse retina and corresponding statistical analysis (B);

FIG. 37 shows the immunofluorescence staining results of IBA1-positive microglia in mouse retinal flat mounts;

FIGS. 38A-C show the results of microglial morphological analysis;

FIGS. 39A-B shows the cytokine Western Blot results (A) and corresponding statistical analysis (B);

FIG. 40 is a schematic diagram of the design for experiment on the effects of intravitreal injection of cGAMP combined with ZOC6 pretreatment on relevant indicators in C57 mice;

FIGS. 41A-B show the Western Blot results (A) and corresponding statistical analysis (B); and

FIGS. 42A-B shows the immunofluorescence staining of blood vessels and red blood cells in retinal flat mounts.

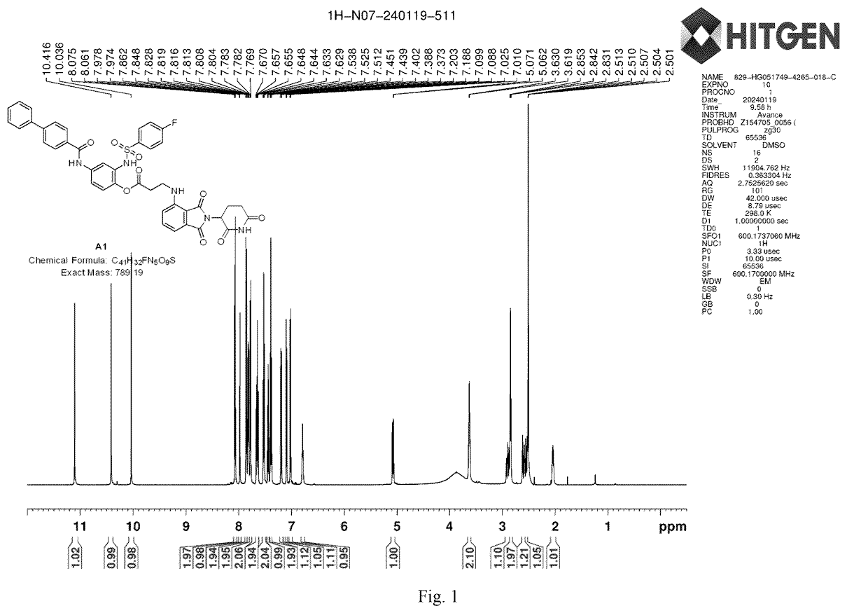

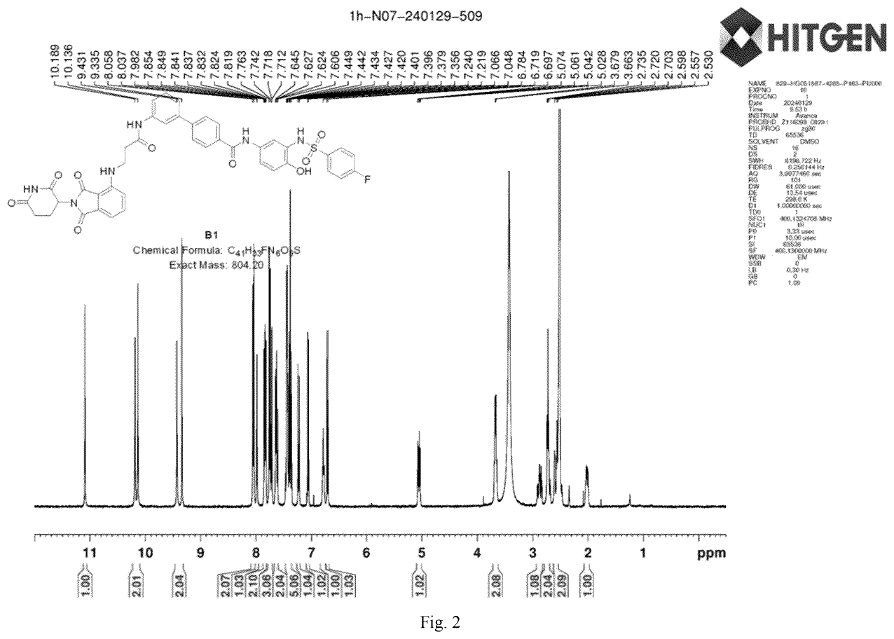

In the figures:

CTL, the abbreviation of “Control”, refers to the reference group without special treatment in an experiment, and is used for comparison with experimental groups (such as those subjected to drug treatment, genetic modification, light damage, etc.);

LD is the abbreviation of “Light Damage”;

BMDM is the abbreviation of Bone Marrow-Derived Macrophage;

TNF-α, abbreviation of tumor necrosis factor-alpha, is an important pro-inflammatory cytokine;

MG132 is a proteasome inhibitor;

WT, the abbreviation of Wild Type, usually refers to the control group with a normal phenotype in its natural state or without artificial intervention (such as gene editing);

DAPI (4′,6-diamidino-2-phenylindole) is a nuclear fluorescent dye for labelling the cell nuclei;

IBA1, the abbreviation of Ionized Calcium-binding Adapter Molecule 1, is used to label microglia;

GFAP is the abbreviation of glial fibrillary acidic protein;

i.vt. (intravitreal injection) means that the drug is directly injected into the vitreous cavity; and

pde6h is the abbreviation of Phosphodiesterase 6H.

### DETAILED DESCRIPTION OF EMBODIMENTS

The raw materials used in the embodiments of the present disclosure can be synthesized according to the methods known in the art, or purchased from Energy Chemical Co., Ltd, Chengdu Kelong Chemical Co., Ltd, Accela ChemBio Co., Ltd, and J&K Scientific Ltd.

Unless otherwise specified, the reaction is performed at room temperature (i.e., 20° C.-30° C.) in a nitrogen atmosphere; the solution refers to an aqueous solution; and M means molar per liter (mol/L).

The compound is structurally characterized by nuclear magnetic resonance (NMR) spectroscopy and mass spectrometry (MS). The NMR shift (δ) is expressed in unit of 10⁻⁶ (ppm), and the NMR analysis is performed on Bruker Avance III 400 or Bruker Avance 600 with dimethyl sulfoxide-d6 (DMSO-d6), deuterated chloroform (CDCl₃) or deuterated methanol (Methanol-d4) as solvent and tetramethylsilane (TMS) as internal standard.

The LC-MS analysis is performed on Shimadzu LC-MS 2020 (ESI), the high-performance liquid chromatography (HPLC) system is Shimadzu LC-20A, and the medium-pressure liquid chromatography (MPLC) system is a reversed-phase preparative chromatography instrument (Gilson GX-281).

The thin-layer chromatography is performed on Huanghai® HSGF254 silica gel plates (Yantai) or GF254 silica gel plates (Qingdao). The thickness of the coating on the silica gel plate for thin-layer chromatography is 0.4 mm to 0.5 mm. Column chromatography generally uses 200-300 mesh silica gel from Yantai Huanghai as the carrier.

TCFH: N,N,N′,N′-tetramethylchloramidine hexafluorophosphate;
NMI: N-methylimidazole;
TEA: Triethylamine;
EDCI: 1-ethyl-(3-dimethylaminopropyl) carbodiimide hydrochloride;
DMAP: 4-(dimethylamino) pyridine;
Pd/C: Palladium on carbon;
(Boc)₂O: di-tert-butyl dicarbonate;
MeCN: Acetonitrile;
MeOH: Methanol;
DMF: N,N-dimethylformamide;
THF: Tetrahydrofuran; and
Found: The MS value displayed when the product is determined by LC-MS.

SP23, the latest STING PROTAC drug (disclosed by Chinese Patent Publication No. 1140852215A), is used as positive control herein. The SP23 is purchased from MedChemExpress LLC (MCE) (#HY-150608), with a structural formula of:

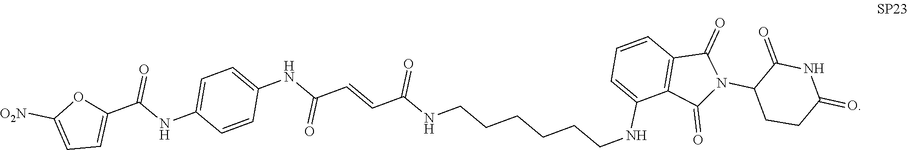 [SMILES: O=C(/C=C/C(=O)Nc1ccc(NC(=O)c2ccc([N+](=O)[O-])o2)cc1)NCCCCCCNc1cccc2c1C(=O)N(C1CCC(=O)NC1=O)C2=O]

The present disclosure will be described in detail below with reference to the embodiments. It should be noted that, the embodiments disclosed herein are merely illustrative, and are not intended to limit the present disclosure.

### EXAMPLE 1 PREPARATION OF COMPOUND A1 (ZOC6)

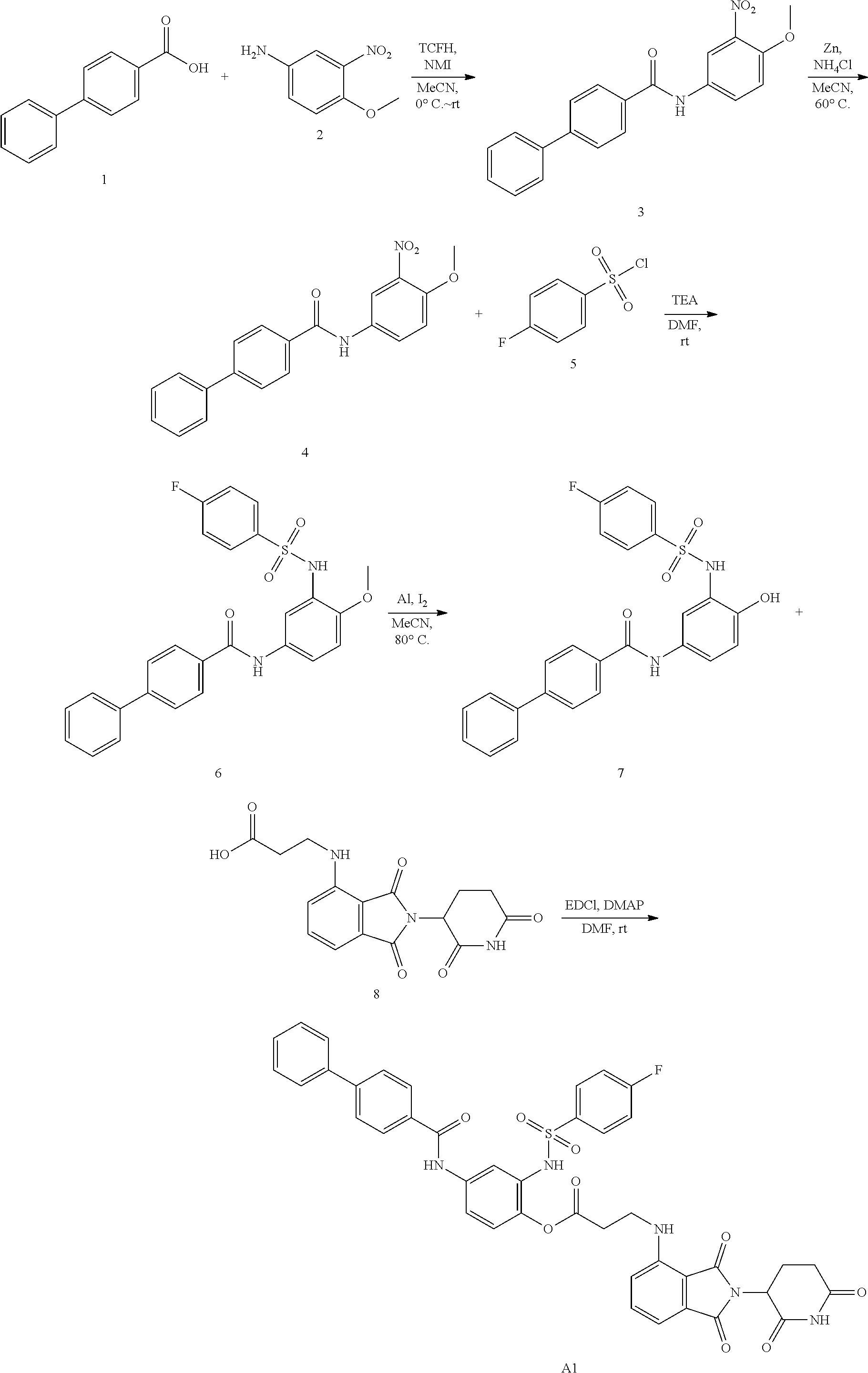

### STEP 1 Preparation of Compound 3

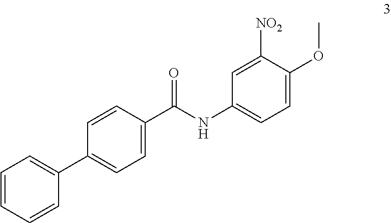 [SMILES: COc1ccc(NC(=O)c2ccc(-c3ccccc3)cc2)cc1[N+](=O)[O-]]

To a 100 mL reaction flask were sequentially added 1.55 g of TCFH (5.55 mmol), 1.06 g NMI (12.61 mmol) and 20 mL of MeCN under an ice bath. The reaction mixture was stirred for 5 minutes, added with 1.00 g of compound 1 (5.04 mmol) and 848.31 mg of compound 2 (5.04 mmol), reacted at room temperature under stirring for 2 hours, and quenched (monitored by LC-MS). The resultant reaction solution was filtered, and the filtered solid was collected, washed with MeCN (3×20 mL) to obtain 1.75 g of crude compound 3.

### STEP 2 Preparation of Compound 4

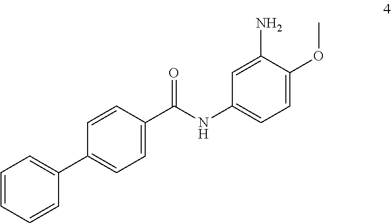 [SMILES: COc1ccc(NC(=O)c2ccc(-c3ccccc3)cc2)cc1N]

To a 100 mL reaction flask were sequentially added 1.25 g of the crude compound 3, 2.67 g of NH₄Cl (49.95 mmol), 3.25 g zinc powder (49.95 mmol) and 30 mL of methanol. The reaction mixture was reacted under stirring at 60° C. for 2 hours, quenched (monitored by LC-MS), and filtered to remove zinc powder. The filtered residue was washed with methanol (2×20 mL). The filtrates were combined, and concentrated under reduced pressure to obtain 1.25 g of crude compound 4.

### STEP 3 Preparation of Compound 6

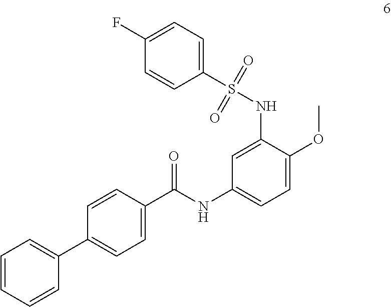 [SMILES: COc1ccc(NC(=O)c2ccc(-c3ccccc3)cc2)cc1NS(=O)(=O)c1ccc(F)cc1]

To a 100 mL reaction flask were sequentially added 1.25 g of the crude compound 4 (3.93 mmol), 916.91 mg of compound 5 (4.71 mmol), 595.95 mg of TEA (821.43 μL, 5.89 mmol) and 20 mL of DMF. The reaction mixture was reacted under stirring at room temperature for 3 hours, quenched with water (monitored by LC-MS), washed with saturated NaCl solution (25 mL) and extracted with ethyl acetate (3×25 mL). The organic phases were combined, dried over anhydrous sodium sulfate, evaporated, purified by column chromatography using DCM/MeOH (100-10:1, v/v) as the eluent and concentrated under reduced pressure to give 650.00 mg of compound 6 (1.36 mmol, 34.74% yield).

### STEP 4 Preparation of Compound 7

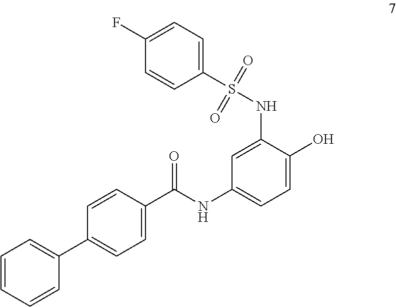 [SMILES: O=C(Nc1ccc(O)c(NS(=O)(=O)c2ccc(F)cc2)c1)c1ccc(-c2ccccc2)cc1]

To a 100 mL reaction flask were sequentially added 650 mg of compound 6 (1.36 mmol), 184.02 mg of A1 (6.82 mmol), 380.83 mg of I₂ (1.50 mmol) and 20 mL of MeCN. The reaction mixture was stirred at 80° C. for 16 hours, quenched with water (monitored by LC-MS), washed with saturated NaCl solution (25 mL) and extracted with ethyl acetate (3×25 mL). The organic phases were combined, dried over anhydrous sodium sulfate evaporated, purified by column chromatography using DCM/MeOH (100-10:1, v/v) as the eluent and concentrated under reduced pressure to give 450.00 mg of compound 7 (972.99 μmol, 71.33% yield).

### STEP 5 Preparation of Compound A1 (ZOC6)

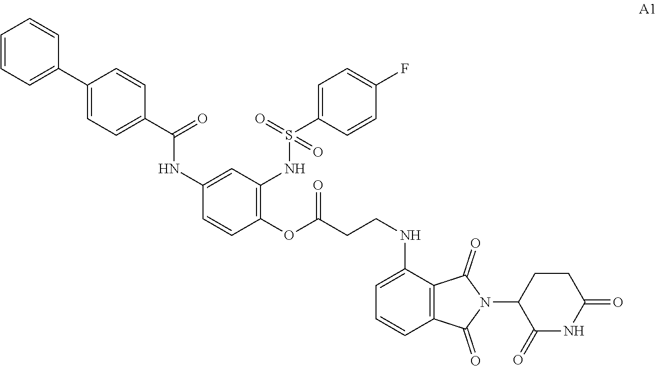 [SMILES: O=C1CCC(N2C(=O)c3cccc(NCCC(=O)Oc4ccc(NC(=O)c5ccc(-c6ccccc6)cc5)cc4NS(=O)(=O)c4ccc(F)cc4)c3C2=O)C(=O)N1]

To a 50 mL reaction flask were sequentially added 46.00 mg of compound 7 (99.46 μmol), 37.78 mg of compound 8 (109.41 μmol), 28.50 mg of EDCI (149.19 μmol), 14.58 mg of DMAP (119.36 μmol) and 5 mL of DMF. The reaction mixture was stirred at room temperature for 16 hours, quenched with water (monitored by LC-MS), washed with saturated NaCl solution (10 mL), and extracted with ethyl acetate (3×10 mL). The organic phases were combined, dried over anhydrous sodium sulfate and evaporated, purified by pre-HPLC, and concentrated under reduced pressure to give 22.7 mg of compound A1 (28.08 μmol, 28.23% yield, 97.7% purity).

LC-MS m/z: [M+H]⁺ calcd. for C₄₁H₃₃FN₅O₉S⁺: 790.19; found: 790.1. ¹H NMR (600 MHz, DMSO-d₆) δ 11.10 (s, 1H), 10.41 (s, 1H), 10.03 (s, 1H), 8.10-8.02 (m, 2H), 7.97 (d, J=2.4 Hz, 1H), 7.88-7.83 (m, 2H), 7.83-7.79 (m, 2H), 7.79-7.74 (m, 2H), 7.68-7.60 (m, 2H), 7.52 (t, J=7.8 Hz, 2H), 7.47-7.41 (m, 1H), 7.40-7.35 (m, 2H), 7.22-7.16 (m, 1H), 7.09 (d, J=6.9 Hz, 1H), 7.01 (d, J=8.7 Hz, 1H), 6.78 (t, J=6.3 Hz, 1H), 5.07 (dd, J=12.9, 5.4 Hz, 1H), 3.62 (q, J=6.6 Hz, 2H), 2.94-2.85 (m, 1H), 2.84 (t, J=6.9 Hz, 2H), 2.64-2.51 (m, 1H), 2.07-1.98 (m, 1H).

Referring to the synthesis method of compound A1 (ZOC6), compounds A2-A6 (ZOC1-5) can be obtained by replacing compound 8 with the raw materials in Table 1 while maintaining other raw materials and operation methods unchanged.

TABLE 1
Compounds A2 to A6

| Compound | | |
|---|---|---|
| No. | Structure and characterization data | Raw material |
| A2 (ZOC1) | 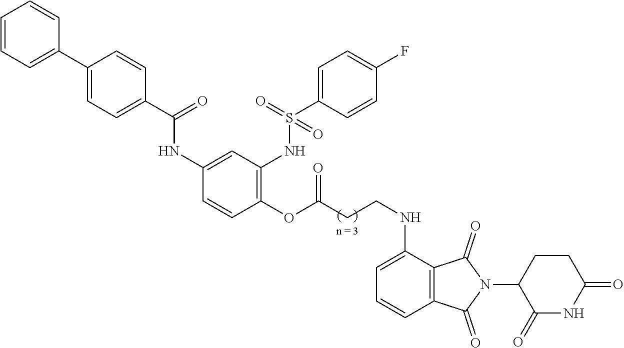 A2 A2 (9.6 mg, 11.73 μmol, 27.12% yield, 99.9% purity). LC-MS m/z: [M + H]⁺calcd. for C₄₃H₃₇FN₅O₉S⁺: 818.22; found: 818.1. ¹H NMR (400 MHz, DMSO-d₆) δ 11.09 (s, 1H), 10.39 (s, 1H), 9.98 (s, 1H), 8.05 (d, J = 8.0 Hz, 2H), 7.93 (d, J = 2.4 Hz, 1H), 7.89-7.73 (m, 6H), 7.64-7.56 (m, 2H), 7.52 (t, J = 7.6 Hz, 2H), 7.41 (dt, J = 17.5, 8.0 Hz, 3H), 7.14 (d, J = 8.6 Hz, 1H), 7.01 (dd, J = 20.4, 7.9 Hz, 2H), 6.62 (t, J = 6.0 Hz, 1H), 5.06 (dd, J = 12.8, 5.4 Hz, 1H), 3.35 (s, 2H), 2.96-2.80 (m, 1H), 2.61 (d, J = 3.2 Hz, 2H), 2.49 (s, 2H), 2.12-1.97 (m, 1H), 1.73-1.56 (m, 4H). | 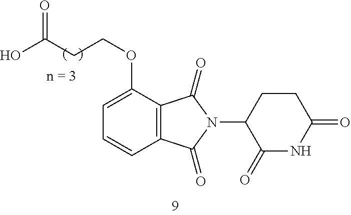 |
| A3 (ZOC2) | 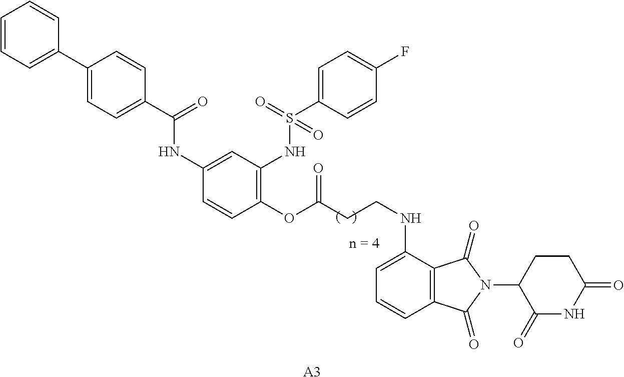 A3 (7.0 mg, 8.41 μmol, 19.44% yield, 99.9% purity). LC-MS m/z: [M + H]⁺calcd. for C₄₄H₃₉FN₅O₉S⁺: 832.24; found: 832.2. ¹H NMR (400 MHz, DMSO-d₆) δ 11.10 (s, 1H), 10.39 (s, 1H), 9.96 (s, 1H), 8.15-8.00 (m, 2H), 7.92 (d, J = 2.4 Hz, 1H), 7.88-7.73 (m, 6H), 7.65-7.55 (m, 2H), 7.52 (dd, J = 8.4, 6.8 Hz, 2H), 7.46-7.34 (m, 3H), 7.13 (d, J = 8.6 Hz, 1H), 7.01 (dd, J = 18.0, 8.0 Hz, 2H), 6.57 (s, 1H), 5.06 (dd, J = 12.8, 5.4 Hz, 1H), 3.33 (d, J = 5.6 Hz, 2H), 2.89 (ddd, J = 17.0, 13.8, 5.4 Hz, 1H), 2.69-2.53 (m, 2H), 2.46 (s, 2H), 2.09-1.97 (m, 1H), 1.62 (tq, J = 15.4, 7.6, 6.8 Hz, 4H), 1.43 (q, J = 7.8 Hz, 2H). | 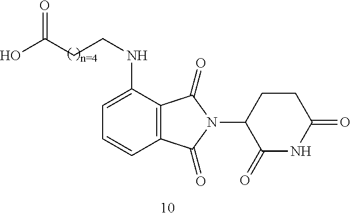 [SMILES: O=C(O)CCNc1cccc2c1C(=O)N(C1CCC(=O)NC1=O)C2=O] |
| A4 (ZOC3) | 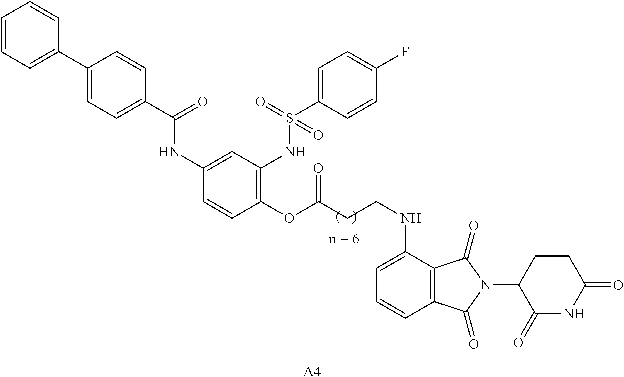 A4 (58.7 mg, 68.19 μmol, 45.06% yield, 99.9% purity). LC-MS m/z: [M + H]⁺calcd. for C₄₆H₄₃FN₅O₉S⁺: 860.27; found: 860.2. ¹H NMR (400 MHz, DMSO-d₆) δ 11.09 (s, 1H), 10.39 (s, 1H), 9.96 (s, 1H), 8.15-8.00 (m, 2H), 7.92 (d, J = 2.4 Hz, 1H), 7.89-7.82 (m, 2H), 7.83-7.74 (m, 4H), 7.63 (dd, J =9.0, 2.4 Hz, 1H), 7.58 (dd, J = 8.6, 7.2 Hz, 1H), 7.52 (dd, J = 8.4, 6.8 Hz, 2H), 7.47-7.35 (m, 3H), 7.13-7.07 (m, 1H), 7.01 (dd, J = 13.8, 8.0 Hz, 2H), 6.55 (s, 1H), 5.05 (dd, J = 12.8, 5.4 Hz, 1H), 3.37-3.26 (m, 2H), 2.88 (ddd, J = 17.4, 14.0, 5.4 Hz, 1H), 2.64-2.52 (m, 2H), 2.44 (t, J = 7.4 Hz, 2H), 2.07-1.98 (m, 1H), 1.66-1.48 (m, 4H), 1.36 (s, 6H). | 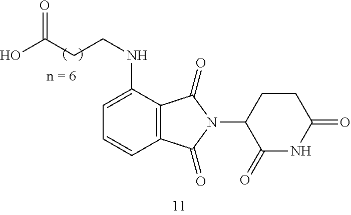 |
| A5 (ZOC4) | 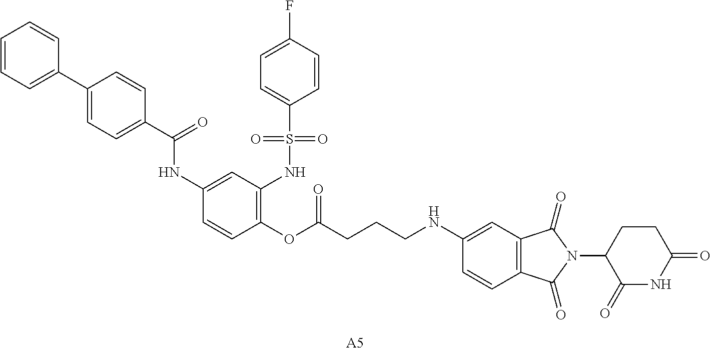 [SMILES: O=C1CCC(N2C(=O)c3ccc(NCCCC(=O)Oc4ccc(NC(=O)c5ccc(-c6ccccc6)cc5)cc4NS(=O)(=O)c4ccc(F)cc4)cc3C2=O)C(=O)N1 ; "A5"] A5 (37.9 mg, 46.54 μmol, 46.79% yield, 98.7% purity). LC-MS m/z: [M + H]⁺ calcd. for C₄₂H₃₅FN₅O₉S⁺: 804.21; found: 804.2. ¹H NMR (400 MHz, DMSO-d₆) δ 11.06 (s, 1H), 10.40 (s, 1H), 10.00 (s, 1H), 8.10-8.03 (m, 2H), 7.94 (d, J = 2.5 Hz, 1H), 7.87-7.74 (m, 6H), 7.66-7.57 (m, 2H), 7.52 (dd, J = 8.3, 6.8 Hz, 2H), 7.46-7.35 (m, 3H), 7.22 (s, 1H), 7.05-6.99 (m, 2H), 6.91 (dd, J = 8.4, 2.1 Hz, 1H), 5.04 (dd, J = 12.9, 5.4 Hz, 1H), 3.27 (s, 2H), 2.88 (ddd, J = 17.2, 14.0, 5.5 Hz, 1H), 2.68-2.52 (m, 4H), 2.06-1.95 (m, 1H), 1.85 (p, J = 7.4 Hz, 2H). | 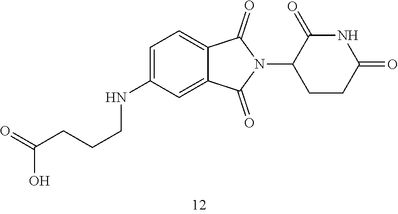 |
| A6 (ZOC5) | 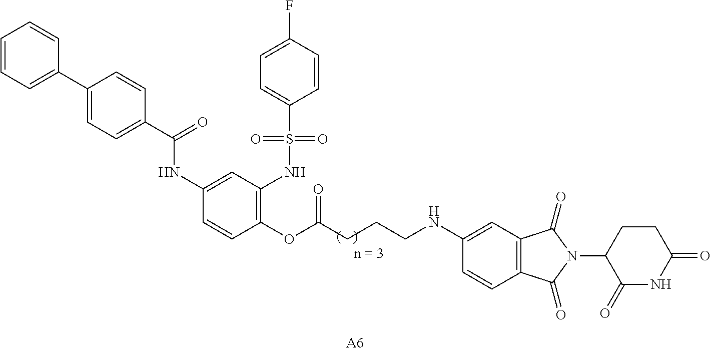 A6 (22.7 mg, 26.17 μmol, 26.20% yield, 95.9% purity). LC-MS m/z: [M + H]⁺ calcd. for C₄₄H₃₉FN₅O₉S⁺: 832.21; found: 832.2. ¹H NMR (600 MHz, DMSO-d₆) δ 11.06 (s, 1H), 10.39 (s, 1H), 9.96 (s, 1H), 8.09-8.03 (m, 2H), 7.92 (d, J = 2.4 Hz, 1H), 7.87-7.82 (m, 2H), 7.82-7.74 (m, 4H), 7.63 (dd, J = 8.7, 2.4 Hz, 1H), 7.57 (d, J = 8.4 Hz, 1H), 7.52 (t, J = 7.8 Hz, 2H), 7.46-7.36 (m, 3H), 7.13 (d, J = 51.3 Hz, 1H), 7.02-6.94 (m, 2H), 6.87 (dd, J = 8.4, 2.1 Hz, 1H), 5.03 (dd, J = 12.9, 5.4 Hz, 1H), 3.20 (t, J = 6.9 Hz, 2H), 2.87 (ddd, J = 16.8, 13.8, 5.4 Hz, 1H), 2.65-2.51 (m, 2H), 2.50-2.45 (m, 2H), 2.03-1.95 (m, 1H), 1.61 (dp, J = 17.4, 7.5 Hz, 4H), 1.49-1.40 (m, 2H). | 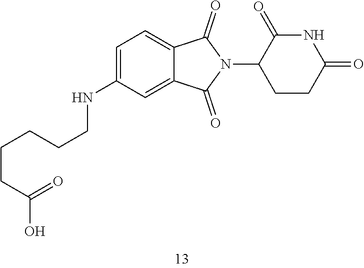 |

### EXAMPLE 2 PREPARATION OF COMPOUND B1 (ZOC7)

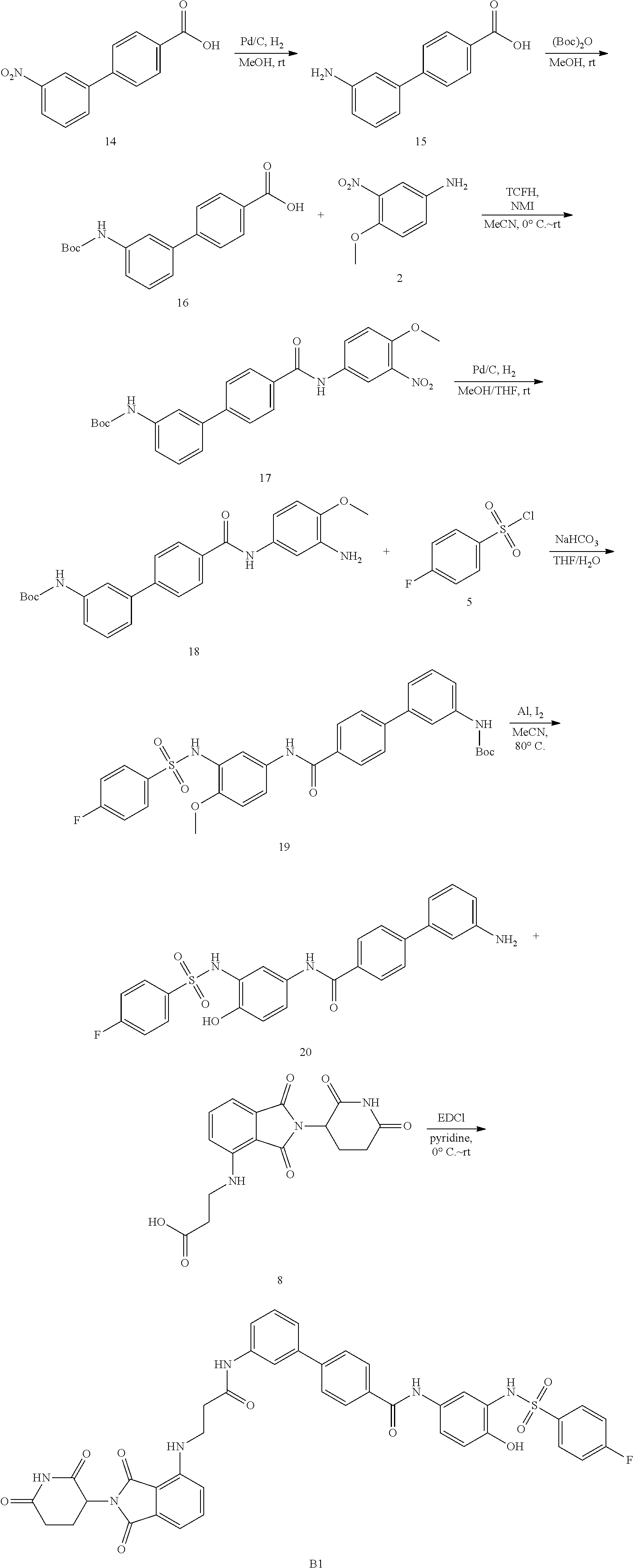

### STEP 1 Preparation of Compound 15

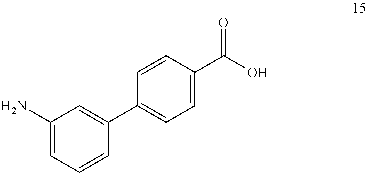 [SMILES: Nc1cccc(-c2ccc(C(=O)O)cc2)c1]

To a 100 mL reaction flask were sequentially added 1.22 g of compound 14 (5.02 mmol), 609.21 mg of Pd/C (5.02 mmol), and 20 mL of methanol. The reaction system was maintained under a hydrogen atmosphere at atmospheric pressure. Then the reaction mixture was stirred at room temperature for 3 hours (monitored by LC-MS). The reaction mixture was filtered, and the Pd/C was washed with methanol (2×20 mL). The filtrates were combined and concentrated under reduced pressure to give 1.10 g of crude compound 15.

### STEP 2 Preparation of Compound 16

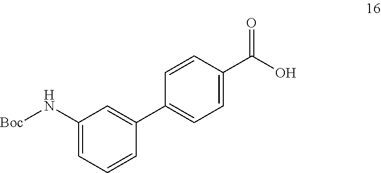 [SMILES: CC(C)(C)OC(=O)Nc1cccc(-c2ccc(C(=O)O)cc2)c1]

To a 100 mL reaction flask were sequentially added 1.10 g of compound 15 (5.16 mmol), 1.12 g of (Boc)₂O (5.16 mmol), 866.98 mg of NaHCO₃ (10.32 mmol) and 20 mL of methanol. The reaction mixture was stirred at room temperature for 16 hours, quenched (monitored by LC-MS). The reaction mixture was extracted with saturated NaCl solution (20 mL) and ethyl acetate (3×20 mL). The organic phases were combined, dried over anhydrous sodium sulfate and concentrated under reduced pressure to give 1.56 g of compound 16.

### STEP 3 Preparation of Compound 17

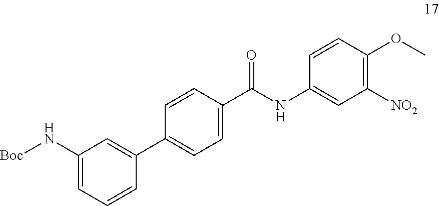 [SMILES: COc1ccc(NC(=O)c2ccc(-c3cccc(NC(=O)OC(C)(C)C)c3)cc2)cc1[N+](=O)[O-]]

To a 100 mL reaction flask were sequentially added 1.67 g of TCFH (5.98 mmol), 1.23 g NMI (14.94 mmol) and 5 mL of MeCN under an ice bath. The reaction mixture was stirred for 5 minutes, added with 1.56 g of compound 16 (4.98 mmol) and 837.13 mg of compound 2 (4.98 mmol), reacted at room temperature under stirring for 2 hours, and quenched (monitored by LC-MS). The resultant solution was extracted with saturated NaCl solution (20 mL) and DCM (3×20 mL). The organic phases were combined, dried over anhydrous sodium sulfate and concentrated under reduced pressure, purified by MPLC to give 2.00 g of compound 17 (4.32 mmol, 86.68% yield).

### STEP 4 Preparation of Compound 18

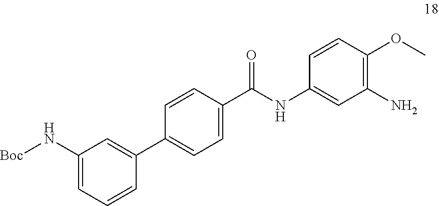 [SMILES: COc1ccc(NC(=O)c2ccc(-c3cccc(NC(=O)OC(C)(C)C)c3)cc2)cc1N]

To a 100 mL reaction flask were sequentially added 2.00 g of compound 17 (4.32 mmol), 524.08 mg of Pd/C (4.32 mmol), and 15 mL of methanol. The reaction system was maintained under a hydrogen atmosphere at atmospheric pressure. Then the reaction mixture was stirred at room temperature for 3 hours and monitored by LC-MS. Further, the reaction mixture solution was filtered out the Pd/C and washed with methanol (2×20 mL). The filtrate was combined and concentrated under reduced pressure to give 1.80 g of crude compound 18.

### STEP 5 Preparation of Compound 19

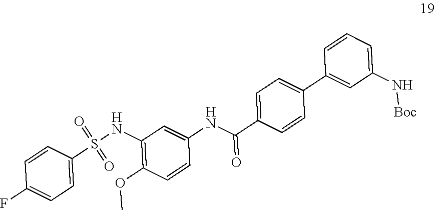 [SMILES: COc1ccc(NC(=O)c2ccc(-c3cccc(NC(=O)OC(C)(C)C)c3)cc2)cc1NS(=O)(=O)c1ccc(F)cc1]

To a 50 mL reaction flask were sequentially added 1.80 g of compound 18 (crude product), 808.08 mg compound 5 (4.15 mmol), 697.58 mg of NaHCO(8.30 mmol), 10 mL of THF and 5 mL of H₂O. The reaction mixture was stirred at room temperature for 16 hours. The reaction mixture was quenched with water (monitored by LC-MS), extracted with saturated NaCl solution (25 mL) and ethyl acetate (3×25 mL). The organic phases were combined, dried over anhydrous sodium sulfate, concentrated under reduced pressure and purified by MPLC to give 2.10 g of compound 19 (3.55 mmol, 85.48% yield).

### STEP 6 Preparation of Compound 20

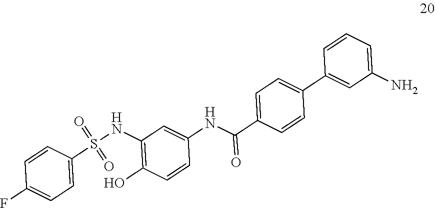 [SMILES: Nc1cccc(-c2ccc(C(=O)Nc3ccc(O)c(NS(=O)(=O)c4ccc(F)cc4)c3)cc2)c1]

To a 50 mL reaction flask were sequentially added 1.00 mg of compound 19 (1.69 mmol), 228.01 mg of A1 (8.45 mmol), 471.89 mg of 12 (1.86 mmol) and 10 mL of MeCN. The reaction mixture was stirred at 80° C. for 16 hours, quenched with water (monitored by LC-MS) and extracted with saturated NaCl solution (25 mL) and ethyl acetate (3×25 mL). The organic phases were combined, dried over anhydrous sodium sulfate, concentrated under reduced pressure and purified by MPLC to obtain 650.00 mg of compound 20 (1.36 mmol, 80.54% yield).

### STEP 7 Preparation of Compound B1 (ZOC7)

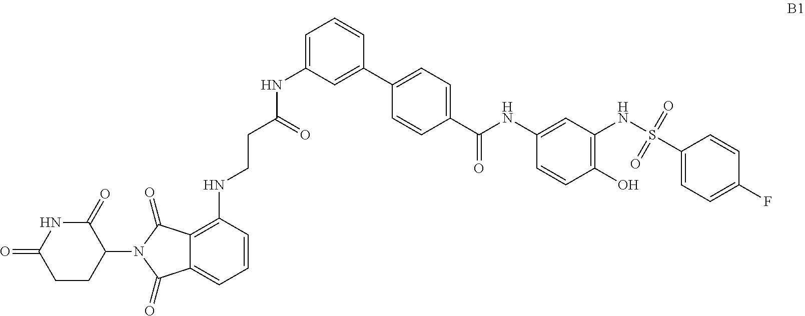 [SMILES: O=C1CCC(N2C(=O)c3cccc(NCCC(=O)Nc4cccc(-c5ccc(C(=O)Nc6ccc(O)c(NS(=O)(=O)c7ccc(F)cc7)c6)cc5)c4)c3C2=O)C(=O)N1]

To a 20 mL reaction flask were sequentially added 34.5 mg of compound 8 (99.91 μmol), 38.17 mg of EDCI (199.82 μmol) and 2 mL of pyridine under an ice bath, added with 47.71 mg of compound 20 (99.91 μmol) after stirring 5 minutes. The reaction mixture was reacted at room temperature under stirring for 2 hours and quenched with water. The resultant solution was extracted with saturated NaCl solution (20 mL) and DCM (3×15 mL). The organic phases were combined, dried over anhydrous sodium sulfate, concentrated under reduced pressure and purified by MPLC to give 2.00 g of compound B1 (25.72 μmol, 25.74% yield, 99.1% purity).

LC-MS m/z: [M+H]⁺ calcd. for C₄₁H₃₄FN₆O₉S⁺: 805.20; found: 805.1. ¹H NMR (400 MHz, DMSO-d₆) δ 11.08 (s, 1H), 10.15 (d, J=21.2 Hz, 2H), 9.38 (d, J=38.8 Hz, 2H), 8.09-8.00 (m, 2H), 7.98 (d, J=2.2 Hz, 1H), 7.87-7.79 (m, 2H), 7.78-7.69 (m, 3H), 7.66-7.58 (m, 2H), 7.46-7.33 (m, 5H), 7.22 (d, J=8.8 Hz, 1H), 7.05 (d, J=7.0 Hz, 1H), 6.78 (t, J=6.2 Hz, 1H), 6.70 (d, J=8.8 Hz, 1H), 5.04 (dd, J=12.8, 5.4 Hz, 1H), 3.66 (q, J=6.4 Hz, 2H), 2.94-2.97 (m, 1H), 2.75-2.63 (m, 2H), 2.63-2.51 (m, 2H), 2.09-1.95 (m, 1H).

Referring to the synthesis method of compound B1 (ZOC7), compounds B2-B4 (ZOC8-10) can be obtained by replacing compound 8 with the raw materials in Table 2 while keeping the other raw materials and operation methods unchanged.

TABLE 2
Compounds of B2 to B4

| Compound | | |
|---|---|---|
| No. | Structure and Characterization data | Raw material |
| B2 (ZOC8) | 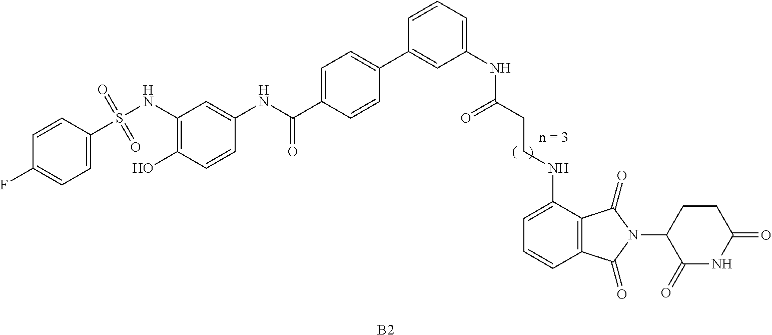 B2 (15.4 mg, 18.49 μmol, 18.51% yield, 93.3% purity). LC-MS m/z: [M + H]⁺ calcd. for C₄₃H₃₈FN₆O₉S⁺: 833.23; found: 833.2. ¹H NMR (400 MHz, DMSO-d₆) δ 11.09 (s, 1H), 10.38 (s, 1H), 9.98 (s, 1H), 8.09-7.99 (m, 2H), 7.93 (d, J = 2.4 Hz, 1H), 7.83-7.77 (m, 2H), 7.77-7.70 (m, 2H), 7.63-7.57 (m, 2H), 7.45-7.36 (m, 2H), 7.25 (dd, J = 15.4, 7.6 Hz, 1H), 7.17-6.94 (m, 6H), 6.80 (d, J = 8.0 Hz, 1H), 6.68-6.57 (m, 1H), 5.06 (dd, J = 12.8, 5.4 Hz, 1H), 3.36 (d, J = 5.6 Hz, 2H), 2.96-2.83 (m, 1H), 2.65-2.52 (m, 4H), 2.12-1.97 (m, 1H), 1.71-1.59 (m, 4H). | 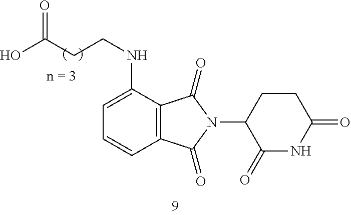 |
| B3 (ZOC9) | 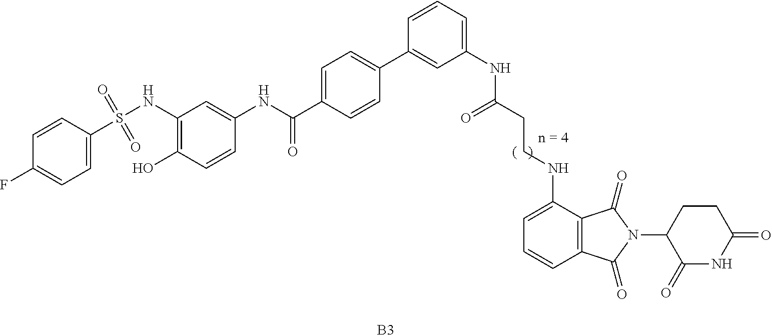 B3 (6.4 mg, 7.56 μmol, 7.57% yield, 89.7% purity). LC-MS m/z: [M + H]⁺ calcd. for C₄₄H₄₀FN₆O₉S⁺: 847.25; found: 847.2. ¹H NMR (400 MHz, DMSO-d₆) δ 11.10 (s, 1H), 10.37 (s, 1H), 9.96 (s, 1H), 8.09-7.98 (m, 2H), 7.91 (d, J = 2.4 Hz, 1H), 7.84-7.76 (m, 2H), 7.76-7.69 (m, 2H), 7.65-7.56 (m, 2H), 7.45-7.33 (m, 2H), 7.29-7.20 (m, 1H), 7.15-6.94 (m, 6H), 6.78 (d, J = 7.8 Hz, 1H), 6.57 (s, 1H), 5.06 (dd, J = 12.8, 5.4 Hz, 1H), 3.34 (d, J = 6.4 Hz, 2H), 2.89 (ddd, J = 17.4, 14.0, 5.4 Hz, 1H), 2.69-2.52 (m, 2H), 2.49-2.41 (m, 2H), 2.11-1.97 (m, 1H), 1.69-1.53 (m, 4H), 1.48-1.37 (m, 2H). | 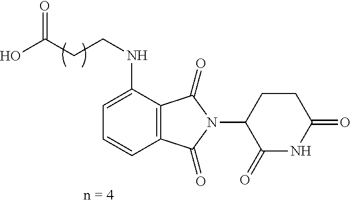 |
| B4 (ZOC10) | 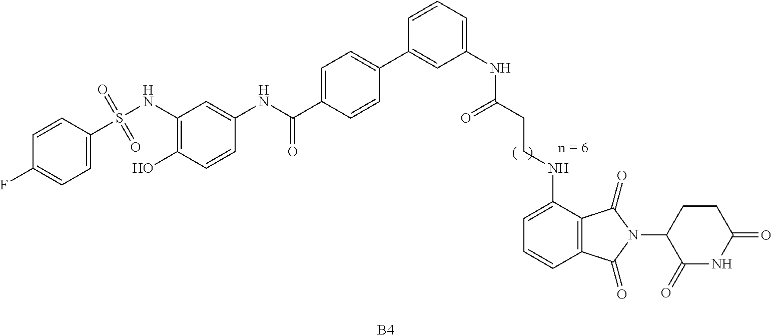 B4 (25.2 mg, 28.80 μmol, 28.83% yield, 96.2% purity). LC-MS m/z: [M + H]⁺ calcd. for C₄₄H₄₀FN₆O₉S⁺: 847.25; found: 847.2. ¹H NMR (400 MHz, DMSO-d₆) δ 11.09 (s, 1H), 10.38 (s, 1H), 9.96 (s, 1H), 8.04 (d, J = 8.4 Hz, 2H), 7.91 (d, J = 2.4 Hz, 1H), 7.83-7.71 (m, 4H), 7.66-7.54 (m, 2H), 7.39 (t, J = 8.8 Hz, 2H), 7.32-6.95 (m, 7H), 6.82 (d, J = 8.0 Hz, 1H), 6.55 (s, 1H), 5.05 (dd, J = 12.8, 5.4 Hz, 1H), 3.32 (d, J = 6.4 Hz, 2H), 2.95-2.76 (m, 1H), 2.69-2.53 (m, 2H), 2.47 (s, 2H), 2.09-1.98 (m, 1H), 1.69- 1.47 (m, 4H), 1.36 (s, 6H). | 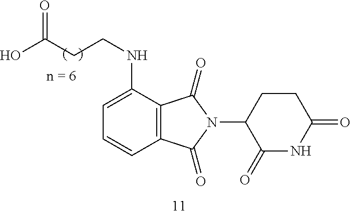 |

The following illustrates the effect of the present disclosure with experiments.

### EXPERIMENTAL EXAMPLE 1 Evaluating the Degradation Degree of STING in Human Retinal Pigment Epithelial Cell Line ARPE-19 by PROTAC Using Western Blot Method

### 1.1 Experimental Instruments and Reagents

SDS-PAGE gel (10%), Cocktail 100× (Selleck), P/S (Gibco), FBS (Nobimpex), 5×loading buffer (EpiZyme), Tween-20, Anti-α-actinin (Proteintech), RIPA, non-fat milk powder (Biofroxx), Anti-STING (CST), 24-well plate (LABSELECT), gel imaging system (Tanon), Immobilon®-PSQ PVDF membrane (Millipore), 1.5 mL centrifuge tube (AXYGEN), Criterion™ Blotter transfer tank (BIO-RAD), DMEM/F12 culture medium (Gibco), and ultrasensitive ECL chemiluminescence kit (NCM).

### 1.2 Experimental Methods

Western Blot analysis: Cells were evenly seeded into 24-well plates and treated with the indicated compounds at corresponding concentrations for 12 hours. Whole-cell lysates were collected using RIPA lysis buffer containing protease inhibitors and phosphatase inhibitors. Protein concentrations were quantified by BCA assay. Equal amounts of proteins were electrophoresed on 10% SDS-PAGE and transferred to polyvinylidene fluoride (PVDF) transfer membranes (0.45 μm). The membranes were incubated with different target antibodies overnight at 4° C. Primary antibodies included α-actinin antibody (Proteintech, #11313-2-AP, 1:5000) and anti-STING antibody (CST, #50494, 1:1000). Corresponding secondary antibodies (1:5000) were incubated at room temperature for 1 hour. The expression level of STING was normalized to that of α-actinin protein. Target bands were exposed and images were recorded. Gray-scale analysis of the bands was performed using imagej.

### 1.3 Data Analysis

Gray-scale analysis software was used for analysis. After calibrating the amount of STING with the amount of α-actinin, the degradation of STING by 10 μM compound was analyzed. Data were presented as mean±standard deviation. Data analysis was performed using one-way ANOVA and Tukey's test. *: P<0.05, **: P<0.01, ***: P<0.001.

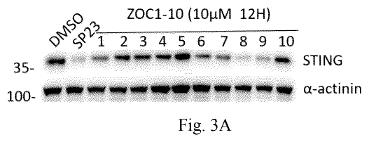

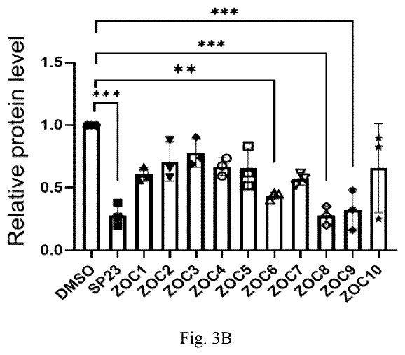

The experimental results are shown in Table 3 and FIGS. 3A-B.

TABLE 3
Degradation of STING in ARPE cells by 10 μM compound
(mean of three replicates, P < 0.05)

| No. | Dₘₐₓ |
|---|---|
| SP23 | 71.8% |
| A2 (ZOC1) | 39.3% |
| A3 (ZOC2) | 29.3% |
| A4 (ZOC3) | 22.3% |
| A5 (ZOC4) | 33.1% |
| A6 (ZOC5) | 34.5% |
| A1 (ZOC6) | 56.5% |
| B1 (ZOC7) | 42.8% |
| B2 (ZOC8) | 72.2% |
| B3 (ZOC9) | 67.7% |
| B4 (ZOC10) | 34.2% |

FIG. 3A showed the results of Western Blot analysis for STING in ARPE cells. As shown in FIG. 3A, compounds ZOC1-10 each exhibited varying degrees of degradation activity against STING in ARPE cells. ZOC6, ZOC8, and ZOC9 demonstrated relatively significant degradation effects, with degradation rates of 56.5%, 72.2%, and 67.7% respectively. SP23, a previously reported effective STING degrader, was utilized as a positive control. FIG. 3B illustrated the statistical results of the grayscale analysis. The results indicated that the degradation effects of compounds ZOC6, ZOC8, and ZOC9 were statistically significant in comparison with DMSO.

### EXPERIMENTAL EXAMPLE 2 Evaluating the Degradation Degree of STING in Human Immune Cell Line THP-1 by PROTAC Using Micro-Content Protein Electrophoresis (WES) Method

### 2.1 Experimental Instruments and Reagents

RIPA, Cocktail 100× (Selleck), Anti-α-actinin (Proteintech), Anti-STING (CST), 24-well plate (LABSELECT), P/S (Gibco), FBS (Nobimpex), RPMI 1640 culture medium (Gibco), and micro-content protein electrophoresis kit (Protein Simple).

### 2.2 Experimental Methods

Cells were evenly seeded into 24-well plates and treated with the indicated compounds at corresponding concentrations for 12 hours before being harvested. Whole-cell lysates were collected using RIPA lysis buffer containing protease inhibitors and phosphatase inhibitors. Protein concentrations were quantified by BCA assay using a BCA protein concentration determination kit. Protein samples with a final concentration of 0.2 μg/μl were used for micro-content protein electrophoresis experiments. The detailed procedures of the micro-content protein electrophoresis experiments were performed in accordance with the instructions provided by the manufacturer (Protein Simple). Primary antibodies included α-actinin antibody (Proteintech, #11313-2-AP, 1:10000) and anti-STING antibody (CST, #50494, 1:100). The expression level of STING was normalized to that of α-actinin protein.

### 2.3 Data Analysis

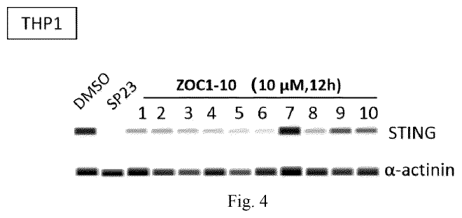

Degradation rates were calculated based on the STING levels. The experimental results are shown in Table 4 and FIG. 4.

TABLE 4
Degradation rates of STING in THP1 cells by 10 μM compounds
(mean of three replicates, P < 0.05)

| Sample Name | Dₘₐₓ |
|---|---|
| SP23 | 87.8% |
| A2 (ZOC1) | 71.4% |
| A3 (ZOC2) | 53.8% |
| A4 (ZOC3) | 47.5% |
| A5 (ZOC4) | 71.1% |
| A6 (ZOC5) | 53.3% |
| A1 (ZOC6) | 76.6% |
| B1 (ZOC7) | 5.6% |
| B2 (ZOC8) | 65.2% |
| B3 (ZOC9) | 26.6% |
| B4 (ZOC10) | 44.4% |

FIG. 4 suggested the results of Western Blot analysis of STING in THP1 cells. As shown in the figure, compounds ZOC1-10 all exhibited varying degrees of degradation effect on STING in THP1 cells. Compound A1 (ZOC6) showed the most significant degradation effect with a degradation rate of 76.6%.

### EXPERIMENTAL EXAMPLE 3 Evaluating the Degradation Degree of STING in Human Neuroblastoma Cell Line SH-SY5Y by PROTAC Using Western Blot

### 3.1 Experimental Instruments and Reagents

SDS-PAGE gel (10%), Cocktail 100× (Selleck), P/S (Gibco), FBS (Nobimpex), 5×loading buffer (EpiZyme), Tween-20, Anti-α-actinin (Proteintech), RIPA, non-fat milk powder (Biofroxx), Anti-STING (CST), 24-well plate (LABSELECT), gel imaging system (Tanon), Immobilon®-PSQ PVDF membrane (Millipore), 1.5 mL centrifuge tube (AXYGEN), Criterion™ Blotter transfer tank (BIO-RAD), DMEM/F12 culture medium (Gibco), ultrasensitive ECL chemiluminescence kit (NCM).

### 3.2 Experimental Methods

Western Blot analysis: Cells were evenly seeded into 24-well plates and treated with the indicated compounds at corresponding concentrations for 12 hours. Whole-cell lysates were collected using RIPA lysis buffer containing protease inhibitors and phosphatase inhibitors. Protein concentrations were quantified by BCA assay. Equal amounts of proteins were electrophoresed on 10% SDS-PAGE and transferred to polyvinylidene fluoride (PVDF) transfer membranes (0.45 μm). For primary antibody incubation, α-actinin antibody (Proteintech, #11313-2-AP, 1:5000) and anti-STING antibody (CST, #50494, 1:1000) were used and incubated overnight at 4° C. For secondary antibody incubation, HRP-conjugated secondary antibody (1:5000) was used and incubated at room temperature for 1 hour. The expression level of STING was normalized to that of α-actinin protein. Target bands were exposed and images were recorded. Gray-scale analysis of the bands was performed using imagej.

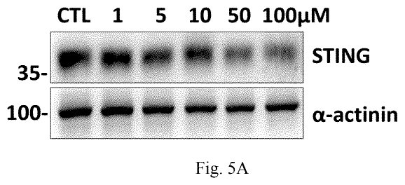

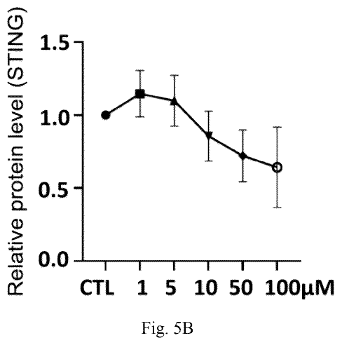

Referring to FIG. 5A showed the results of Western Blot analysis of STING in SH-SY5Y cells, and FIG. 5B showed the statistical results of grayscale analysis. The figures suggested that the degradation of STING in SH-SY5Y cells by compound ZOC6 exhibited a concentration-dependent manner. The degradation rate at 100 μM was 36%.

### EXPERIMENTAL EXAMPLE 4 ARPE Cytotoxicity Experiment

### 4.1 Experimental Instruments and Reagents

ARPE (human retinal pigment epithelial cells), 48-well plate/96-well plate (Costar), Calcein/PI Cell Viability and Cytotoxicity Detection Kit (Beyotime, C2015S), Cell Counting Kit-8 (Beyotime, C0037), high-content cell imaging system (ImageXpress Micro 4, PerkinElmer), microplate reader (Synergy HTX, Biotek).

### 4.2 Experimental Methods

### 4.2.1 Live/Dead Cell Staining

seeding and culture: Cells were seeded in 48-well plates and treated with corresponding drugs for 6 hours;

washing: The culture medium was aspirated, and cells were washed once with PBS;

staining: Calcein AM/PI detection working solution was prepared (Calcein AM 1:1000, PI 1:1000). 150 μL of Calcein AM/PI detection working solution was added to each well, followed by incubation at 37° C. for 1 hour; and

detection: After incubation, images were captured using a high-content cell imaging system.

### 4.2.2 Cell Viability Detection by CCK8 (Cell Counting Kit-8) Assay

seeding and culture: ARPE cells were seeded in 96-well plates and treated with corresponding drugs for 12 hours;

washing: The culture medium was aspirated, and cells were washed once with PBS;

incubation: CCK8 detection working solution was prepared (CCK8: medium=10:100). 100 μL of CCK8 detection working solution was added to each well, followed by incubation at 37° C. for 2 hours; and

detection: After incubation, the absorbance was measured at 450 nm using a microplate reader.

### 4.3 Data Analysis

$$Cell viability ( \% ) = [ ( A s - Ab ) / ( Ac - A b ) ] \times 100 \% ;$$

As: Absorbance of the experimental group (containing cells, medium, CCK-8 solution, and drug solution);

Ac: Absorbance of the control group (containing cells, medium, CCK-8 solution, without drug); and

Ab: Absorbance of the blank group (containing medium, CCK-8 solution, without cells or drug).

Data are presented as mean±standard deviation. Data analysis was performed using two-way ANOVA followed by Tukey's test.

SP23, a latest STING PROTAC drug with the published patent application number CN1140852215A, was used as the positive control. The SP23 (positive control) was purchased from MCE (#HY-150608).

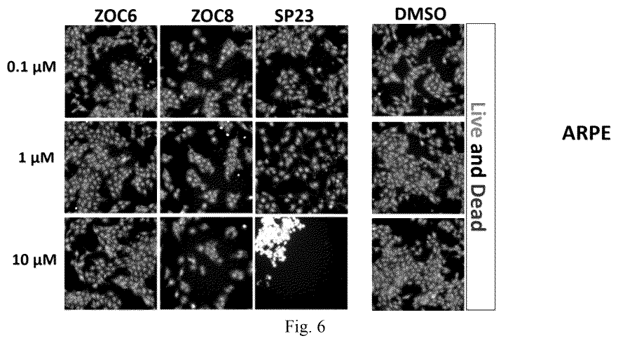

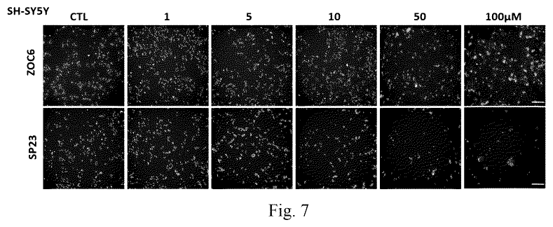

FIG. 6 showed the results of live/dead cell staining of ARPE cells, in which green represents live cells and red represents dead cells. SP23 served as the positive control. The results in FIG. 6 indicated that there was no significant difference in the number of dead cells or cell morphology compared with the DMSO group under treatment with ZOC6 and ZOC8 at different concentrations (0.1, 1, 10 μM). In contrast, SP23 at 1 μM significantly increased the number of dead cells. Further, 10 μM SP23 exhibited greater toxicity to ARPE cells, resulting in massive cell death and altered cell morphology (aggregation).

The statistical results of live/dead cell staining of ARPE cells are shown in Table 5.

TABLE 5
Statistical results of live/dead cell staining
of ARPE cells (mean of three replicates)

| | | Percent of Live Cell |
|---|---|---|
| Sample Name | Concentration (μM) | (%) |
| DMSO | 0.1 | 98.9 |
| | 1 | 97.5 |
| | 10 | 95.8 |
| SP23 | 0.1 | 98 |
| | 1 | 83.8 |
| | 10 | 12.7 |
| A1 (ZOC6) | 0.1 | 98.7 |
| | 1 | 98.4 |
| | 10 | 96.2 |
| B2 (ZOC8) | 0.1 | 99.4 |
| | 1 | 97.7 |
| | 10 | 97 |

Table 6 showed the experimental data of cell viability detected by the CCK8 assay. These data indicated that under the conditions of 10 μM and 12-hour treatment, the cytotoxicity of the compounds mentioned in the table was more than 3 times lower than that of SP23. At 10 μM, the cell survival rate after SP23 treatment was 28.375%, suggesting that SP23 has high cytotoxicity. In contrast, after treatment with A1 (ZOC6), B2 (ZOC8), and B3 (ZOC9) at 10 μM, the cell survival rates were 92.3%, 94.34%, and 92% respectively (p<0.05), indicating that even at high concentrations, the treatment with A1 (ZOC6), B2 (ZOC8), and B3 (ZOC9) did not affect cell viability, and the compounds exhibited low cytotoxicity.

TABLE 6
Statistical results of cell viability detected
by the CCK8 assay (mean of three replicates)

| Sample Name | Concentration (μM) | Percent of Live Cell (%) |
|---|---|---|
| SP23 | 0 | 100 |
| | 0.1 | 91.395 |
| | 1 | 89.69 |
| | 10 | 28.375 |
| A1 (ZOC6) | 0 | 100 |
| | 0.1 | 98.57 |
| | 1 | 92.83 |
| | 10 | 92.3 |
| B2 (ZOC8) | 0 | 100 |
| | 0.1 | 96.49 |
| | 1 | 95.5 |
| | 10 | 94.34 |
| B3 (ZOC9) | 0 | 100 |
| | 0.1 | 95.69 |
| | 1 | 91.65 |
| | 10 | 92 |

### EXPERIMENTAL EXAMPLE 5 SH-SY5Y Cytotoxicity Experiment

SH-SY5Y cells were seeded in 48-well plates. After 24 hours, they were treated with compound ZOC6 at different concentrations (1-100 μM) or SP23 at different concentrations (1-100 μM) for 12 hours. The culture medium was discarded, and the cells were washed twice with PBS. A dual-staining working solution containing calcein-AM (1:1000) and propidium iodide (PI, 1:1000) was added, followed by incubation at 37° C. in the dark for 30 minutes. Observations and counting were performed under a fluorescence microscope.

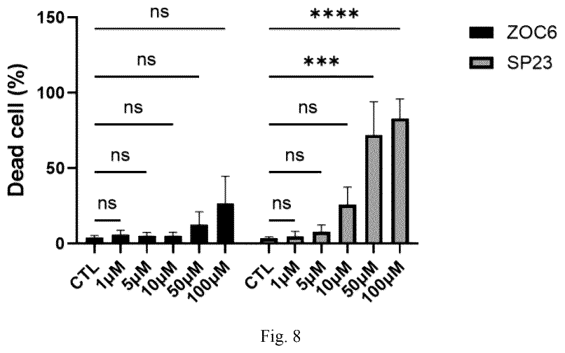

FIG. 7 presented the results of live/dead cell staining of SH-SY5Y cells. The results showed that under treatment with ZOC6 and SP23 at different concentrations, SP23 increased the number of dead cells at 10 μM, leading to massive cell death, altered cell morphology, and cell aggregation. The statistical results are shown in FIG. 8. At 10 μM SP23, the number of dead cells exceeded 25%; SP23 at 50 μM (72%, p<0.005) and 100 μM (83%, p<0.001) caused a significant increase in the number of dead cells. In contrast, at 10 μM ZOC6, the number of dead cells was only 5%, which was more than 5 times lower than that of SP23. Even at higher concentrations, the number of dead cells in the ZOC6-treated groups showed no significant difference compared with the control group.

### EXPERIMENT 6 Detection of the Effects of ZOC6 and SP23 on Mouse Retinal Function Via Intravitreal Injection of Compounds

Numerous studies have shown that high expression of STING caused by retinal damage leads to the loss of visual function in mice (PMID: 35347235, PMID: 37217935). In this embodiment, the effect of ZOC6 on visual function was evaluated through Electroretinogram (ERG) experiments, and compared with SP23 to clarify its toxic and side effects in the eye.

### 6.1 Experimental Instruments and Reagents

5-8-week-old C57BL/6J mice (Experimental Animal Center of Sun Yat-sen University, Guangzhou), 0.1% sodium pentobarbital, compound tropicamide (Zhuobian), hydroxypropyl methylcellulose eye drops (Affiliated Eye Hospital of Sun Yat-sen University), insulin needles (BD), microsyringes (Hamilton), ERG measuring instrument (Diagnosys Celeris).

### 6.2 Experimental Methods

### 6.2.1 Intravitreal Injection Administration

The mice were anesthetized with 1% sodium pentobarbital, and compound tropicamide eye drops were used to dilate the pupils of the mice; hydroxymethyl cellulose gel was used to lubricate the cornea. For intravitreal injection, an insulin needle was first used to make a small opening at the limbus corneae, preferably without bleeding, then a microsyringe was used to slowly insert the needle at an angle of about 45 degrees along the opening at the limbus corneae. The needle should not be inserted too deeply to avoid puncturing the retina or scratching the lens. The drug was slowly pushed in. After injection, the needle was kept in the vitreous cavity for about 5 seconds to prevent drug leakage, and then the needle was slowly withdrawn.

### 6.2.2 Retinal Function Analysis

Two days after intravitreal injection, the mice were anesthetized with 0.1% sodium pentobarbital. After mydriasis with tropicamide, the corneas of the mice were coated with hydroxypropyl methylcellulose for moisture retention. The mice were placed on the ERG instrument (Diagnosys Celeris), and two electrodes were in contact with the two corneas of the mice respectively, and the mice were stimulated with flashes of different intensities.

### 6.2.3 Mouse Vision Detection

The visual function of mice was detected using the stripe instrument (STRIA TECH Optodrum Plus, STRIA TECH). The optokinetic response stabilizes the image on the retina by observing moving environments/objects and turning the head (eye movement) (the optokinetic reflex is responsible for eye movements related to the same target). The visual ability (vision) of animals is evaluated by observing their head movements (black and white stripes) to adjust the moving environment. The visual behavior of the mice was not artificially interfered with.

The experiment used C57BL/6J mice, with intravitreal injection of 1 μL of 100 μM ZOC6 or SP23, and the vision of the mice was measured 3 days after injection. Throughout the process, the mice were kept in a completely closed box, provided with a constant rotation speed (12°/s) and a constant black and white stripe contrast (99.72%). The staircase method was used to determine the spatial frequency (cyc/deg), ranging from 0.056 to 0.50 cycles per degree, and the maximum spatial frequency that the mice could pass through was measured. The maximum spatial frequency value that can be passed through reflects the visual acuity of the mice; the larger the value, the smaller the stripes that the mice can distinguish, and the better the visual acuity. The inspection was first performed clockwise, then counterclockwise. Four monitors were used to simulate the stripes, and the mice were observed through a camera above the box. The instrument's software used a special algorithm to measure the scores of the mice under different parameters.

### 6.2.4 Hematoxylin-Eosin Staining of Paraffin Sections of Major Organs

### (1) Paraffin Sectioning

fixation: after the mice were euthanized, the eyeballs were removed and placed in PBS to remove excess connective tissue, then placed in 1 mL of FAS eyeball fixative for overnight fixation.

dehydration: the fixed eyeballs were dehydrated in 60%, 70%, 80%, 90%, and 100% ethanol for 1 hour each, then a small opening was made at the cornea, and the eyeballs were placed in new 100% ethanol for 40 minutes of dehydration.

clearing: the dehydrated eyeballs were placed in a solution of xylene: 100% ethanol=1:1 for 10 minutes, then placed in xylene I and II for 15 minutes each.

wax Impregnation: the eyeballs were impregnated in low-melting-point wax I for 2 hours, low-melting-point wax II for 1 hour, and high-melting-point wax III for 3 hours.

embedding: first, the tissue embedding frame was preheated by immersion in high-melting-point wax. After taking it out, a thin layer of wax was first added to the bottom to hold the eyeball. The eyeball was placed in the horizontal direction of the corneal optic nerve. After the lower layer of wax solidified, the remaining wax was slowly added to fill the embedding frame, which was then placed on a cold stage to stand until the wax was completely solidified.

sectioning: a paraffin microtome was used for sectioning, and sagittal sections of the eyeball were made with a thickness of 8 μm. The sections were placed in a 42° C. water bath for spreading, and then mounted on glass slides. The mounted slides were marked and stored at room temperature in a slide box for later use.

### (2) Hematoxylin-Eosin (HE) Staining

slide baking: the paraffin sections were placed in a 60° C. oven for 30 minutes to fully melt the paraffin.

deparaffinization: the baked slides were placed in xylene I, II, and III for 10 minutes each.

rehydration: the slides were placed in 100% ethanol twice, 5 minutes each time, then sequentially placed in 95%, 90%, 80%, and 70% ethanol for gradient rehydration, 3 minutes each, and washed with ddH₂O 3 times, 3 minutes each time.

hematoxylin staining: the sections were placed in hematoxylin staining solution for 5 minutes, and after staining, they were soaked in pure water for 1 minute.

differentiation: the sections were placed in hydrochloric acid alcohol for differentiation for 10 seconds, then rinsed with running water 3 times, 3 minutes each time.

eosin staining: the sections were placed in eosin staining solution for 1 minute, then soaked in pure water for 1 minute.

dehydration: after staining, the sections were dehydrated again, first soaked in 95% alcohol for 30 seconds, then repeated with new 95% alcohol once, then soaked in 100% alcohol for 2 minutes, and the process was repeated with new 100% alcohol once.

clearing: after dehydration, clearing was performed, and the sections were placed in xylene I, II, and III for 10 minutes each.

mounting: mounting was done with neutral gum.

observation: images were captured and analyzed using the bright field of the TissueFAXs panoramic tissue cell imaging quantitative analysis system.

### 6.3 Data Analysis

The data were presented as mean±standard deviation, and the data analysis was performed using two-way ANOVA and Tukey's test. *: P<0.05.

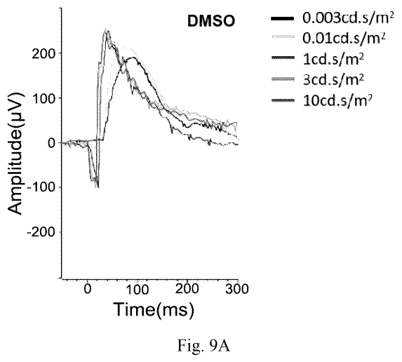

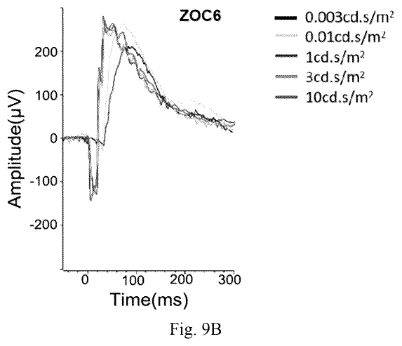

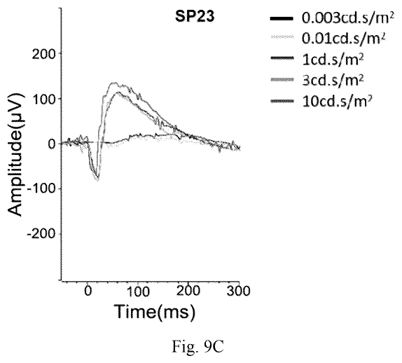

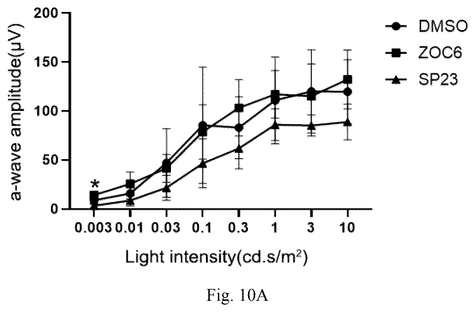

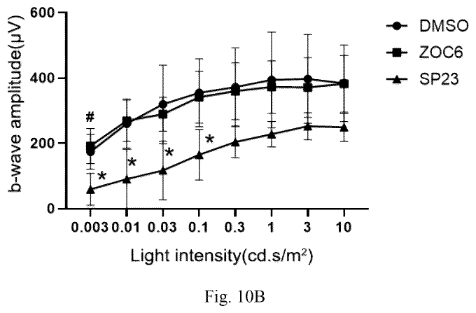

The experimental results are shown in Table 7, FIGS. 9A-C, and FIGS. 10A-B.

Electroretinogram (ERG) was used to analyze retinal function and observe the toxic effects of intravitreally injected compounds on the eyes of mice. FIGS. 9A-C showed representative ERG waveforms of mice in each treatment group, and FIGS. 10A-B showed statistical graphs of the amplitudes of ERG a-waves and b-waves in mice under different intensities of flash stimulation. As shown in FIGS. 9A-C and FIGS. 10A-B, compared with the control DMSO group and the ZOC6 treatment group, the amplitudes of a-wave and b-wave in the SP23-injected group were decreased under different flash stimulations, while there was no significant change in the amplitudes of a-wave and b-wave between the ZOC6 treatment group and the control DMSO group. Under the intensity of 0.003 cd·s/m², the differences in the amplitudes of a-wave and b-wave between ZOC6 and SP23 were statistically significant; under the intensities of 0.01, 0.03, and 0.1 cd-s/m², the differences in the amplitude of b-wave between ZOC6 and SP23 were statistically significant. In general, the response threshold of mice to flash stimulation increased after SP23 injection, indicating that the visual function of mice in the SP23-injected group was poorer than that in the DMSO group and the ZOC6 group, and compound SP23 has certain toxicity to the retina.

TABLE 7
Results of Mouse ERG Experiment

| Intensity of flash | | | | | |
|---|---|---|---|---|---|
| stimulation | | | | | Mean Value |
| (cd · s/m²) | DMSO (a-wave) | | | | (a-wave) |
| 0.003 | 4.132 | 8.008 | 14.18 | 9.153 | 8.86825 |
| 0.01 | 22.63 | 9.085 | 14.07 | 18.05 | 15.95875 |
| 0.03 | 31.66 | 99.25 | 32.68 | 24.51 | 47.025 |
| 0.1 | 64.27 | 173.7 | 60.12 | 44.26 | 85.5875 |
| 0.3 | 53.49 | 109.9 | 110.7 | 57.94 | 83.0075 |
| 1 | 96.31 | 133.4 | 157.6 | 56.19 | 110.875 |
| 3 | 99.13 | 148.1 | 162.1 | 70.84 | 120.0425 |
| 10 | 91.57 | 136.7 | 157.5 | 93.45 | 119.805 |
| Intensity of flash | | | | | |
| stimulation | | | | | Mean Value |
| (cd · s/m²) | ZOC6 (a-wave) | | | | (a-wave) |
| 0.003 | 8.624 | 15.8 | 15.21 | 17.69 | 14.331 |
| 0.01 | 9.837 | 27.66 | 26.28 | 39.36 | 25.78425 |
| 0.03 | 20.48 | 48.6 | 50.96 | 46.13 | 41.5425 |
| 0.1 | 37.77 | 97.76 | 87.5 | 91.98 | 78.7525 |
| 0.3 | 60.23 | 116.7 | 117.6 | 118.7 | 103.3075 |
| 1 | 82.36 | 119.5 | 129.4 | 137.1 | 117.09 |
| 3 | 69.88 | 113.7 | 132.6 | 144.7 | 115.22 |
| 10 | 87.26 | 147 | 144.6 | 150.4 | 132.315 |
| Intensity of flash | | | | | |
| stimulation | | | | | Mean Value |
| (cd · s/m²) | SP23 (a-wave) | | | | (a-wave) |
| 0.003 | 4.968 | 5.392 | 0.443 | 2.638 | 3.36025 |
| 0.01 | 10.81 | 3.713 | 5.073 | 15.51 | 8.7765 |
| 0.03 | 8.872 | 21.85 | 17.15 | 39.13 | 21.7505 |
| 0.1 | 26.53 | 54.17 | 27.1 | 78.59 | 46.5975 |
| 0.3 | 44.88 | 67.9 | 46.2 | 88.8 | 61.945 |
| 1 | 82.2 | 73.24 | 79.33 | 109.6 | 86.0925 |
| 3 | 85.42 | 76.4 | 79.06 | 100.3 | 85.295 |
| 10 | 86.01 | 81.31 | 73.37 | 114.9 | 88.8975 |
| Intensity of flash | | | | | |
| stimulation | | | | | Mean Value |
| (cd · s/m²) | DMSO (b-wave) | | | | (a-wave) |
| 0.003 | 183.4 | 241.5 | 159.1 | 112.7 | 174.175 |
| 0.01 | 239 | 340.1 | 296.8 | 165.9 | 260.45 |
| 0.03 | 263.6 | 454.3 | 377.7 | 183 | 319.65 |
| 0.1 | 349.5 | 428.3 | 431.3 | 209.5 | 354.65 |
| 0.3 | 321.8 | 449.8 | 489.8 | 227 | 372.1 |
| 1 | 348.8 | 472.7 | 544.1 | 210.6 | 394.05 |
| 3 | 354.3 | 478.9 | 529.8 | 226.4 | 397.35 |
| 10 | 334.9 | 449.6 | 506.9 | 243.5 | 383.725 |
| Intensity of flash | | | | | |
| stimulation | | | | | Mean Value |
| (cd · s/m²) | ZOC6 (b-wave) | | | | (a-wave) |
| 0.003 | 113 | 202.5 | 224.9 | 227.9 | 192.075 |
| 0.01 | 181.9 | 268 | 296.5 | 330.1 | 269.125 |
| 0.03 | 212.9 | 304.3 | 320.1 | 319.8 | 289.275 |
| 0.1 | 227.2 | 367.7 | 360.9 | 409.9 | 341.425 |
| 0.3 | 232.6 | 391.9 | 389.5 | 425.1 | 359.775 |
| 1 | 256.4 | 390.4 | 400.9 | 442.3 | 372.5 |
| 3 | 241 | 389.5 | 399.1 | 454.5 | 371.025 |
| 10 | 253.7 | 406.8 | 424.2 | 444.3 | 382.25 |

| Intensity of flash | | | | | |
|---|---|---|---|---|---|
| stimulation | | | | | Mean Value(a- |
| (cd · s/m²) | SP23 (b-wave) | | | | wave) |
| 0.003 | 86.58 | 114.2 | 14.94 | 21.89 | 59.4025 |
| 0.01 | 164.6 | 174.4 | 12.45 | 11.48 | 90.7325 |
| 0.03 | 186.4 | 199.8 | 61.85 | 20.58 | 117.1575 |
| 0.1 | 224.2 | 233.9 | 129.7 | 73.05 | 165.2125 |
| 0.3 | 243.2 | 248.1 | 171.5 | 155 | 204.45 |
| 1 | 269.8 | 252.5 | 190.5 | 199.4 | 228.05 |
| 3 | 288.9 | 261.8 | 193.1 | 266.9 | 252.675 |
| 10 | 304.8 | 237.9 | 201.1 | 253.6 | 249.35 |

The results in Table 7 show that intraocular injection of ZOC6 did not affect the function of retinal cells, and thus intraocular injection could be used in the treatment of ophthalmic diseases in subsequent applications.

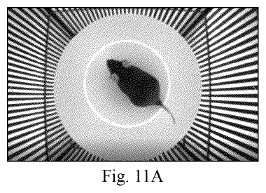

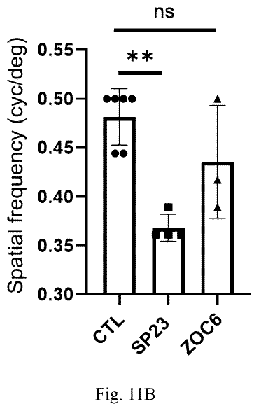

FIGS. 11A-B show the schematic diagram of the mouse vision detection experiment process (A) and the statistical analysis of the results (B). CTL represents the solvent control group. As shown in the results, intravitreal injection of SP23 (100 μM) significantly impairs mouse vision (p<0.01 vs. CTL control group), while the vision of mice treated with ZOC6 at the same concentration (100 μM) showed no significant difference compared with the solvent control group.

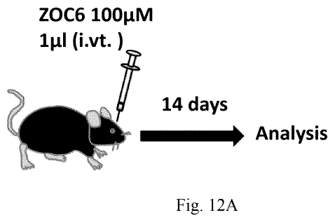

FIG. 12A is a schematic diagram of the experimental design. 1 μL of ZOC6 (100 μM) was injected intravitreally, and samples were collected for experiments after 14 days of long-term observation to evaluate the toxic effects of intravitreal injection of ZOC6. FIG. 12B shows the statistical results of mouse weight changes over 14 days. There was no significant difference in weight changes between the mice in the intravitreal ZOC6 injection group and those in the solvent control injection group.

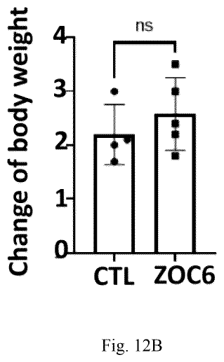

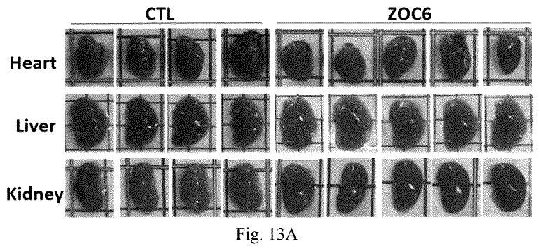

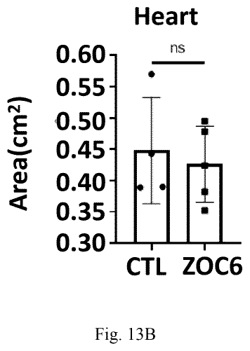

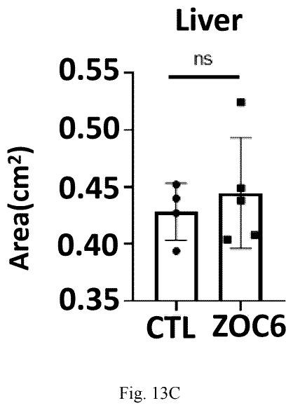

FIG. 13A shows the gross images of major organs (heart, liver, kidney) of mice 14 days after intravitreal injection of ZOC6 (100 μM). The statistical results of the size measurement of major organs are shown in FIG. 13B, indicating no significant difference in organ size between the solvent control group and the intravitreal ZOC6 injection group.

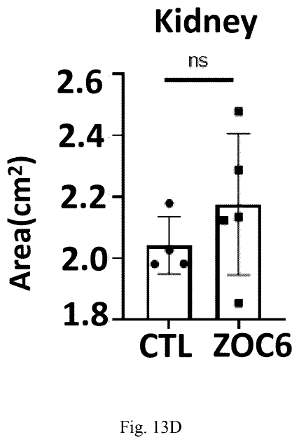

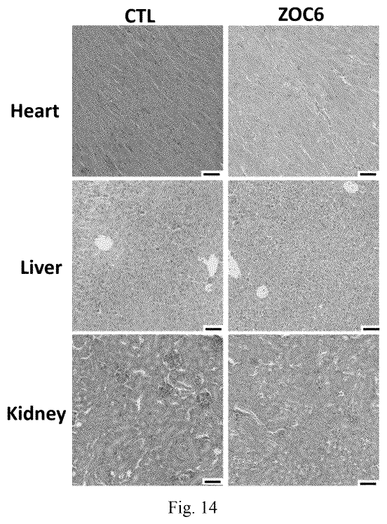

FIG. 14 presents the H&E-stained images of paraffin sections of major organs (heart, liver, kidney) of mice 14 days after intravitreal injection of ZOC6 (100 μM). It shows that there is no significant difference in organ structure between the blank control group and the intravitreal ZOC6 injection group.

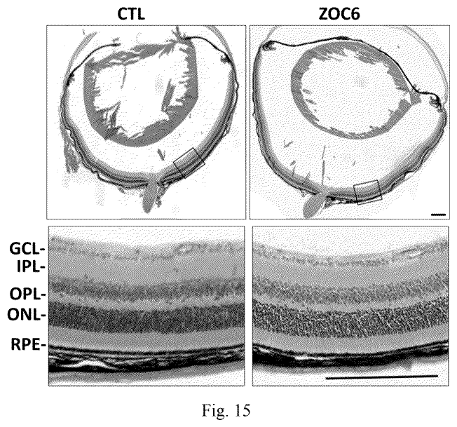

FIG. 15 showed the H&E-stained images of paraffin sections of mouse eyes 14 days after intravitreal injection of ZOC6. It indicated that there was no significant difference in eye structure between the blank control group and the intravitreal ZOC6 injection group.

### EXPERIMENTAL EXAMPLE 7 Evaluation of the Inhibitory Effect of PROTAC on STING-Downstream Inflammatory Responses Via Western Blot, Enzyme-Linked Immunosorbent Assay, and Immunofluorescence Assay

(1) 2′-3′cGAMP is a second messenger that triggers STING activation and a commonly used STING agonist. In this study, liposomes were directly used to transfect cGAMP into cells to activate STING, aiming to detect the inhibitory effect of PROTAC on STING-downstream inflammatory responses.

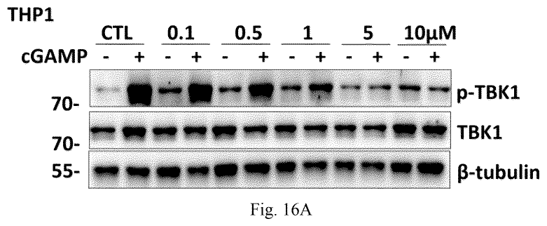

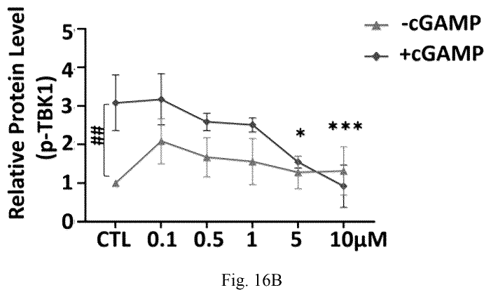

The human monocytic leukemia cell line THP1 was used in the experiment. THP1 cells were treated with ZOC6 at specified concentrations (0.1-10 μM) for 12 hours, followed by transfection with 1 μg/μL cGAMP to activate the STING signal pathway. The phosphorylation level of downstream TBK1 (TANK-binding kinase 1) was detected by WB to reflect the inhibitory effect of PROTAC (ZOC6) on STING-downstream inflammatory responses. The results are shown in FIGS. 16A-B. Among them, p-TBK1 refers to the phosphorylated form of TBK1 protein, and its phosphorylation status usually reflects the activity level of this kinase. TBK1 refers to the total TBK1 protein (including phosphorylated and non-phosphorylated forms). β-tubulin is used as a reference protein. The numbers (70 and 55) indicate the molecular weight of the protein.

FIGS. 16A-B showed the Western Blot results (A) and grayscale statistical analysis (B). In the figures, −cGAMP indicates no cGAMP transfection, and +cGAMP indicates cGAMP transfection. The results showed that cGAMP transfection increased the phosphorylation level of TBK1, and ZOC6 treatment significantly inhibited TBK1 phosphorylation with an IC50 of 1.35 μM.
(2) Bone marrow-derived macrophages (BMDMs) are commonly used to evaluate the activation of the STING pathway in-vitro. IRF3 is also an effector molecule downstream of STING, and changes in its nuclear localization reflect its activation state. In general, STING activation promotes IRF3 nuclear translocation.

BMDM cells were used in the experiment. BMDM cells were treated with 10 μM ZOC6 for 12 hours, then transfected with 1 μg cGAMP to activate STING, and the cells were fixed for immunofluorescence assay 6 hours later.

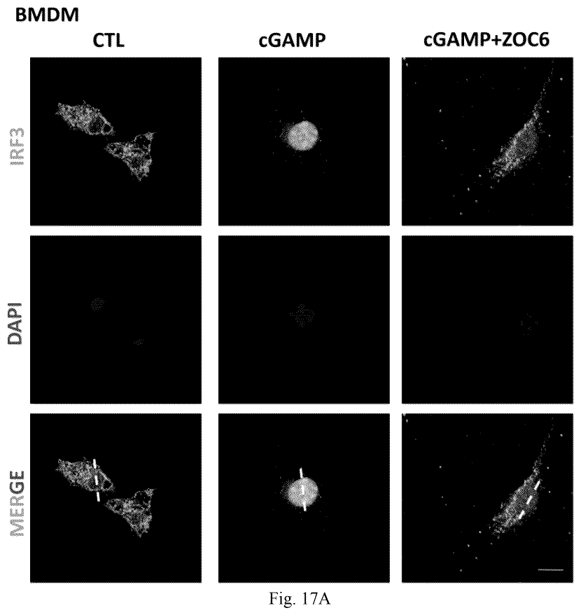

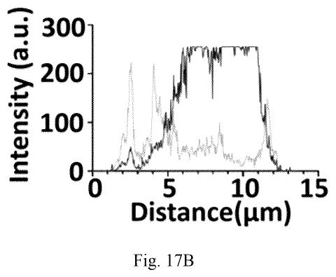

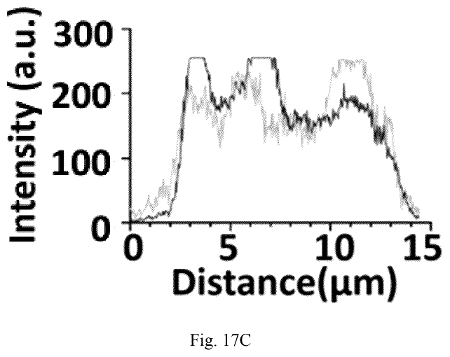

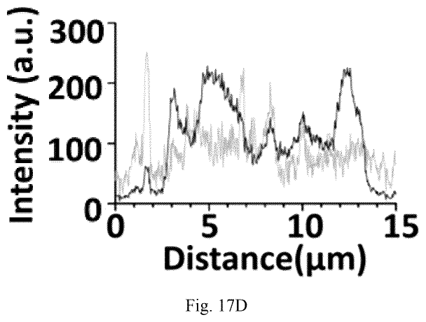

FIGS. 17A-D showed the immunofluorescence results. As shown in FIG. 17A, the nuclear signal (blue) and IRF3 signal (green) highly overlap after cGAMP transfection, indicating that cGAMP treatment induces IRF3 translocation to the nucleus, while after ZOC6 treatment, the IRF3 signal (green) mainly exists in the cytoplasm. FIG. 17B is a co-localization analysis diagram of immunofluorescence, where the horizontal axis is the position and the vertical axis is the fluorescence intensity. The higher the overlap of the two curves, the closer the spatial positions of the two molecules. The figure showed that the blue nuclear signal and green IRF3 signal highly overlap after cGAMP treatment, indicating that cGAMP treatment induces IRF3 translocation to the nucleus and activates STING downstream, while the overlap of the two curves decreases after ZOC6 treatment (cGAMP+ZOC6), indicating that ZOC6 inhibits STING downstream activation.
(3) IL6 and TNFα are inflammatory factors produced after STING activation, and their levels are often used to reflect the activation of the STING signal pathway.

Mouse bone marrow-derived macrophages (BMDMs) were used in the experiment. BMDMs were treated with 10 μM ZOC6 for 12 hours, then transfected with cGAMP to activate STING, and the supernatant of cell lysate was collected 6 hours later to detect the contents of IL6 and TNFα.

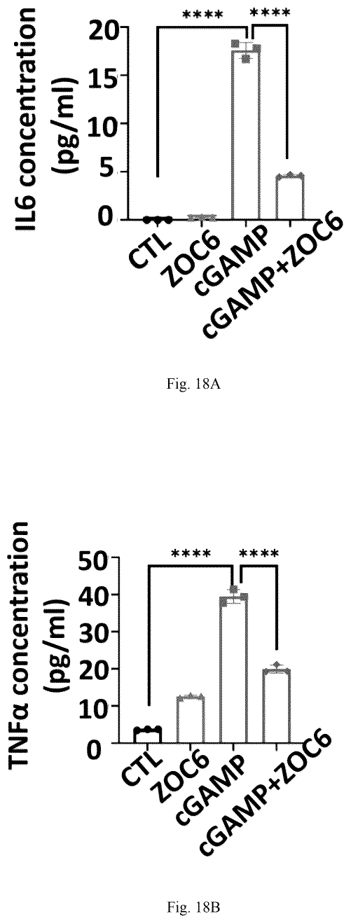

FIGS. 18A-B showed the results of the enzyme-linked immunosorbent assay. As shown in FIGS. 18A-B, cGAMP treatment significantly induces the production of IL6 (mean: 17.6 μg/ml, p<0.0001) and TNFα (mean: 39.5 μg/ml, p<0.0001), while ZOC6 (p<0.001) can significantly inhibit the production of IL6 and TNFα.

### EXPERIMENTAL EXAMPLE 8 Evaluation of the Specificity of PROTAC-Mediated STING Degradation Via Western Blot and Enzyme-Linked Immunosorbent Assay

PROTAC (Proteolysis-Targeting Chimera) degrades target proteins through the ubiquitin-proteasome system. To evaluate the specificity of PROTAC-mediated STING degradation, this study treated cells with the ubiquitin-proteasome inhibitor MG132, followed by detection of STING levels in the collected cells.

MG132 (a proteasome inhibitor), also known as Z-LLL-CHO, Z-Leu-Leu-Leu-CHO, is a commonly used proteasome inhibitor. It has a molecular weight of 475.62 Da and a CAS number of 133407-82-6.

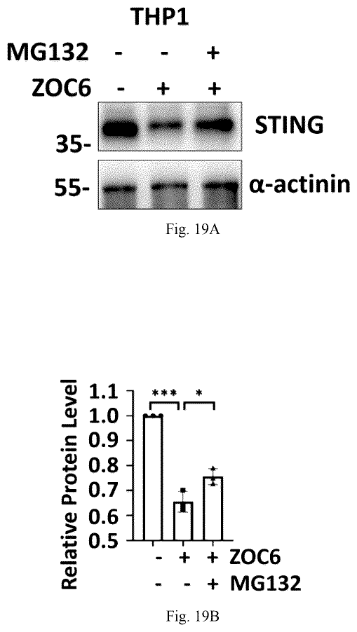

FIGS. 19A-B showed the Western Blot results (A) and corresponding statistical analysis (B). In the abscissa, “−” indicates no inhibitor added, and “+” indicated inhibitor addition. The results demonstrated that MG132 could partially inhibit ZOC6-mediated STING degradation (p<0.05).

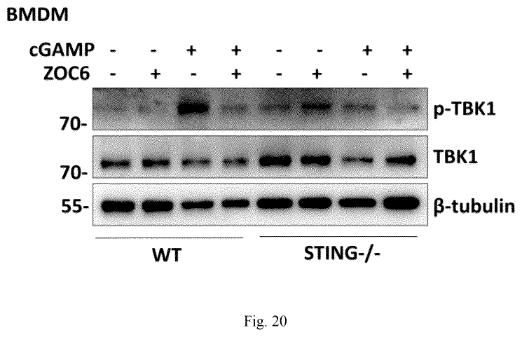

FIG. 20 presented the Western Blot results. To assess the inhibitory effect of PROTAC (ZOC6) on STING downstream inflammatory responses, experiments were performed using wild-type (WT) and STING knockout (STING^{-/-}) BMDM (bone marrow-derived macrophage) cells. Cells were treated with 10 μM ZOC6 for 12 hours, then transfected with 1 μg cGAMP to activate the STING pathway. The phosphorylation level of downstream TBK1 was detected by Western Blot (WB). As shown in FIG. 20, in wild-type BMDMs, cGAMP transfection increased TBK1 phosphorylation, and ZOC6 treatment significantly inhibited TBK1 phosphorylation. However, such cGAMP-induced elevation of TBK1 phosphorylation and its inhibition by ZOC6 were not observed in STING knockout BMDMs. These findings indicated that the inhibitory effect of PROTAC (ZOC6) on the STING-TBK1 signaling pathway is specific and dependent on the presence of STING.

FIG. 21 showed the statistical results of the Western Blot in FIG. 20. cGAMP transfection significantly increased TBK1 phosphorylation (p<0.001), and ZOC6 treatment significantly inhibited TBK1 phosphorylation (p<0.01).

### EXPERIMENTAL EXAMPLE 9 To Effectively Inhibit the Systemic Inflammatory Response in TREX1−/− Mice Using Intraperitoneal Injection of ZOC6

Trex1 is a key DNA exonuclease, whose main function is to degrade abnormal or excessive DNA in the cytoplasm. When the Trex1 gene is knocked out or mutated, mice are unable to effectively clear these cytoplasmic DNA, leading to abnormal accumulation of endogenous DNA in the cytoplasm. Eventually, this results in the continuous activation of STING, triggering autoimmune responses in multiple tissues and organs. Therefore, Trex1-deficient mice are the gold standard model for evaluating therapeutic strategies targeting the cGAS or STING pathways (such as inhibitors). Testing the drug on Trex1 knockout mice can clearly assess whether the drug can effectively inhibit the overactivated STING pathway, reduce interferon levels, and alleviate inflammation and tissue damage.

In the embodiment, Trex1 gene knockout mice with a C57BL/6J background and wild-type mice with a C57BL/6J background were intraperitoneally injected with 10 mg/kg ZOC6 once a day for 7 consecutive days. On the 10th day, after the first administration, the mice were anesthetized and sacrificed, and multiple organ tissue samples were collected and sectioned. The sections were subjected to HE staining and immunofluorescence observation.

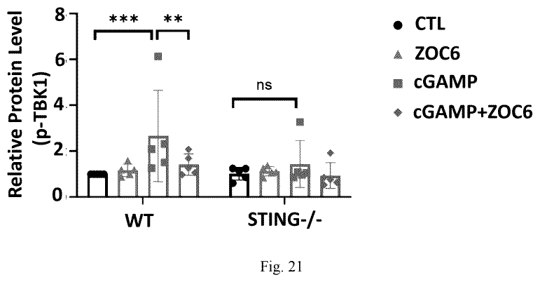

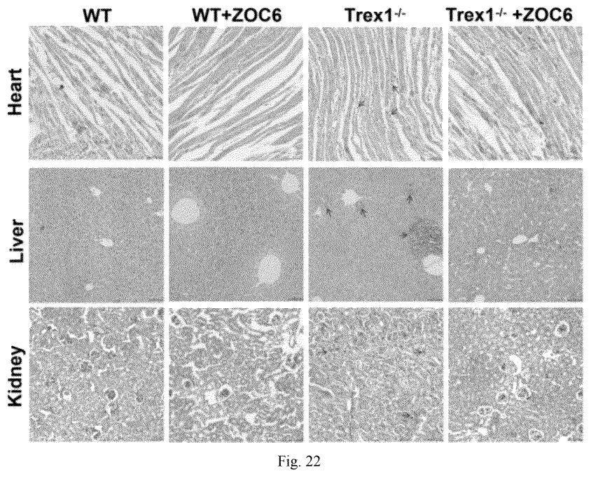

FIG. 22 showed the HE staining of the paraffin sections of the main organs (heart, liver and kidney) of the mice. As shown in the figure, TREX1 knockout mice had different degrees of inflammatory cell infiltration in these organs, especially in the liver, where a large number of inflammatory cell clusters could be seen, and the red arrows were the infiltrated inflammatory cells.

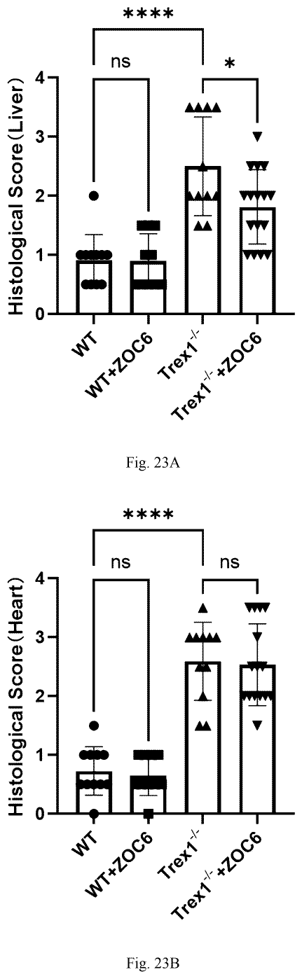

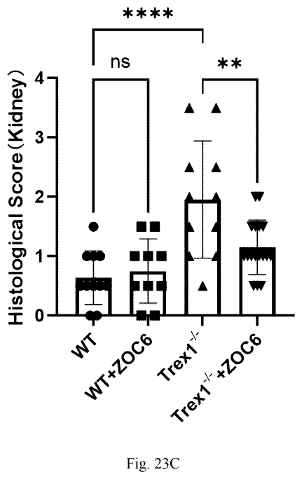

FIGS. 23A-C showed the inflammation statistics of HE staining, with 0 points representing no inflammatory cell infiltration and 4 points representing severe infiltration. Compared with wild-type mice (WT group), TREX1 knockout mice (Trex−/− group) had significant inflammatory cell infiltration in heart, liver and kidney (p<0.0001), while ZOC6 (Trex−/−+ZOC6 group) could partially alleviate liver (p<0.05), kidney (p<0.01).

FIG. 24 showed the immunofluorescence staining results of IBA1 (Ionized Calcium-binding Adapter Molecule 1) positive microglia in mouse retinal paraffin sections. The increase in the number of microglia in the retina and their morphological changes (towards amoeba-like transformation) were both markers of retinal inflammatory responses. As shown in the figure, activated, amiba-like microglia were present in the retina of TREX1 knockout mice (Trex−/− group), and the number of IBA1-positive (green fluorescent labeled) microglia significantly increased, indicating that the absence of TREX1 led to excessive activation of retinal microglia. The treatment with ZOC6 (Trex−/−+ZOC6 group) could reduce the number of IBA1-positive microglia and decrease the activation degree of microglia, suggesting that ZOC6 treatment could effectively inhibit the retinal inflammatory response caused by TREX1 deficiency.

staining and labeling description: DAPI (4′,6-diamidino-2-phenylindole, 4′,6-diamidino-2-phenylindole), whose blue fluorescence is used to label the cell nucleus, showing the distribution of retinal tissue cells; and

MERGE (Merging Channels): to superimpose IBA1 (green) and DAPI (blue) signals can visually display the spatial location and distribution density of microglia in retinal tissue.

FIG. 25 showed the immunofluorescence staining results of GFAP (Glial Fibrillary Acidic Protein) in paraffin sections of mouse retina. The increased expression and altered localization of GFAP in the retina are markers of astrocyte activation and also one of the markers of retinal inflammatory response. As shown in the figure, compared with the wild-type control group, the GFAP signal (green fluorescent labeling) in the retina of TREX1 knockout mice (Trex−/− group) was significantly enhanced, while the treatment with ZOC6 (Trex−/−+ZOC6 group) could alleviate the GFAP signal in the retina of TREX1 knockout mice. It was suggested that ZOC6 treatment could effectively inhibit the activation of astrocytes caused by TREX1 deletion and alleviate retinal inflammatory responses.

staining and labeling description: DAPI: Blue fluorescently labeled cell nuclei, showing the distribution and stratification of retinal tissue cells; and

MERGE (Merge Channel): Superimpose GFAP (green) and DAPI (blue) signals to visually display the location and distribution of GFAP positive cells in retinal tissue.

The above results indicate that intraperitoneal injection of ZOC6 can effectively inhibit the overactivated STING pathway, reduce interferon levels, and alleviate inflammation and tissue damage.

### EXPERIMENTAL EXAMPLE 10 to Degrade STING in the Light-Induced Retinal Injury Model with ZOC6 and to Alleviate Inflammatory Responses as Well as Further Protect the Retina

To verify the degradation effect of ZOC6 on STING in animals, this experiment adopted the reported retinal injury model induced by light damage that can activate STING. The experiment used balb/c mice because the mice in this background lack melanin and are more sensitive to strong light, which is conducive to model establishment. Before light injury, mice received a single intravitreal injection of 1 μl of ZOC6 with a concentration of 100 μM. Subsequently, the mice were exposed to an LED of 15,000 Lux for 2 hours to induce retinal injury and establish a retinal injury model. Materials were collected three days after modeling for the observation of retinal morphology and other related experiments.

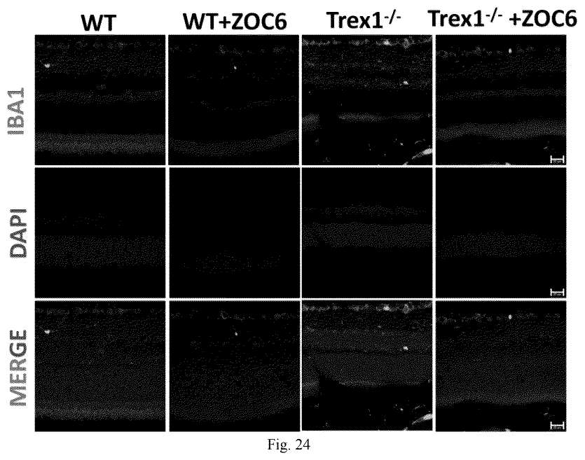

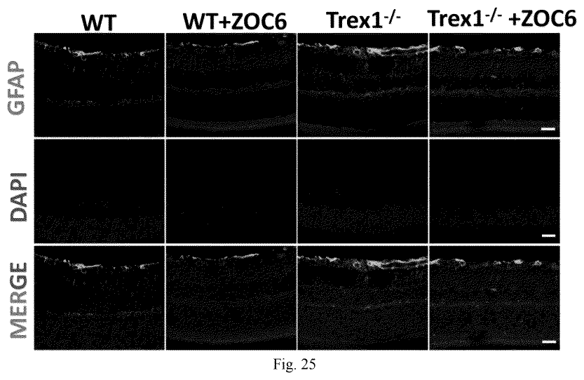

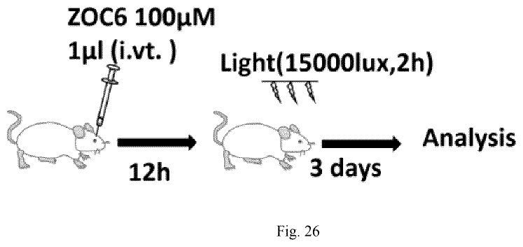

FIG. 26 is a schematic diagram of the experimental design. In the subsequent related experiments, CTL was the blank group (without light damage), the ZOC6 group was intravitreal injection of ZOC6 but without light damage treatment, the LD+DMSO group was represented by intravitreal injection of solvent+light damage, and the LD+ZOC6 group was represented by intravitreal injection of ZOC6+light damage.
the English abbreviations in the picture include:
Ganglion Cell Layer (GCL);
Inner Nuclear Layer (INL);
Outer Plexiform Layer (OPL); and
Outer Nuclear Layer Retinal Pigment Epithelium (RPE)).

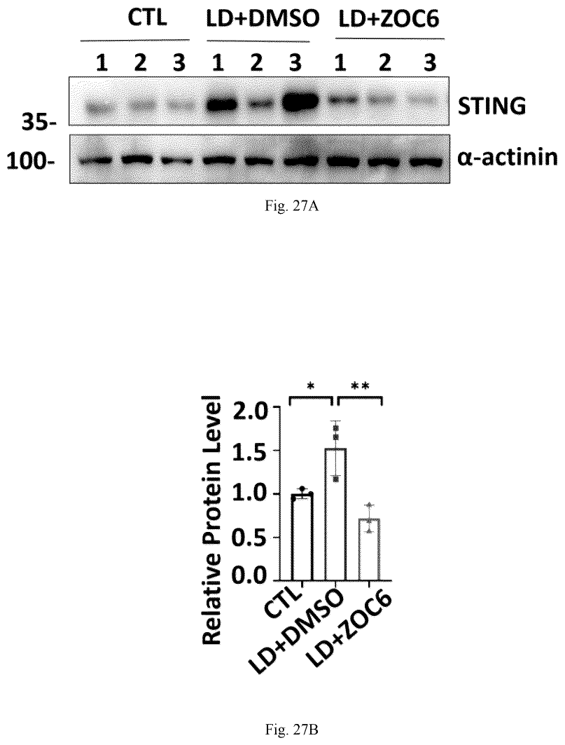

FIGS. 27A-B showed the Western Blot results (A) and the corresponding statistical analysis (B). In FIG. 27A, the numbers 1, 2, and 3 represented three independent biological replicates (from different mice), and the numbers (35 and 100) indicate the protein molecular weight. The Width and gray scale of the bands represented the protein quantity. The experimental results showed that there are certain differences in the degree of light damage in each mouse, so there are differences in the activation degree of STING and the protein expression level of STING. Overall, the expression level of STING after light damage was higher than that of the blank control group. FIG. 27B showed the protein quantification results. The expression level of STING was normalized based on the expression level of α-actinin protein. Gray-scale analysis of the bands was conducted using imagej. As shown in the figure, light damage (LD+DMSO group) could cause an increase in the expression level of STING in the retina (p<0.05), while ZOC6 (LD+ZOC6 group) could degrade the elevated STING caused by light damage (p<0.01).

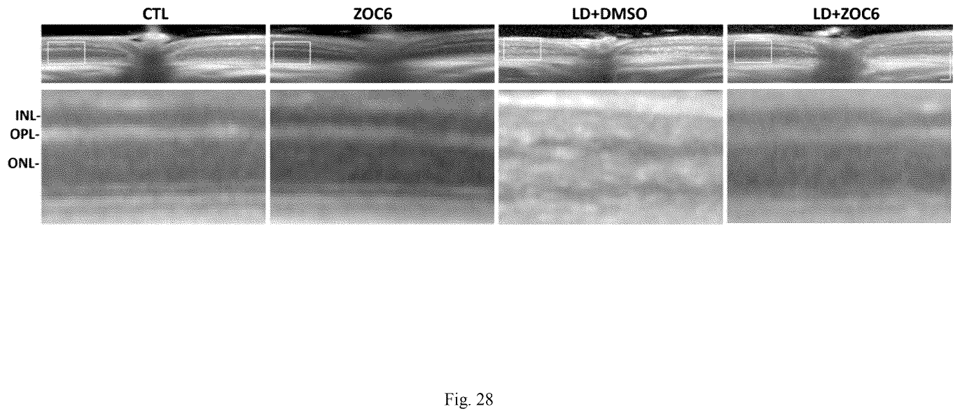

FIG. 28 showed a scan of optical coherence tomography (OCT), a non-contact, high-resolution imaging technique used for in vivo imaging of the posterior segments of the eye, including the retina, retinal nerve fiber layer, etc. As shown in the figure, light damage caused a pathological phenomenon of hyperreflection in the outer nuclear layer of the retina, which was alleviated by ZOC6 treatment. It suggested that intravitreal injection of ZOC6 can protect the retina to a certain extent.

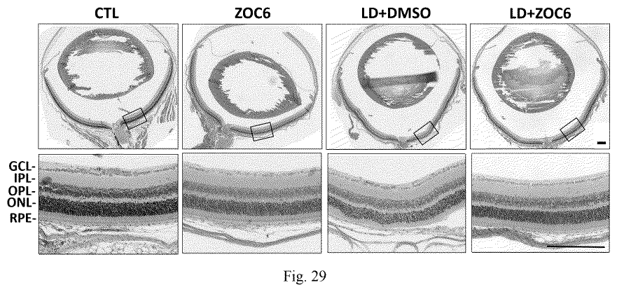

FIG. 29 showed HE staining of paraffin sections of the mouse retina. As shown in the figure, light damage caused disordered arrangement and thinning of cells in the outer nuclear layer of the retina, which was alleviated after ZOC6 treatment.

FIGS. 30A-B showed TUNEL staining of the mouse retina (A) and corresponding statistical analysis (B), which was used to detect apoptotic cells. As shown in the figure, light damage caused apoptosis of retinal photoreceptors, while ZOC6 treatment significantly inhibited photoreceptor apoptosis (p<0.01).

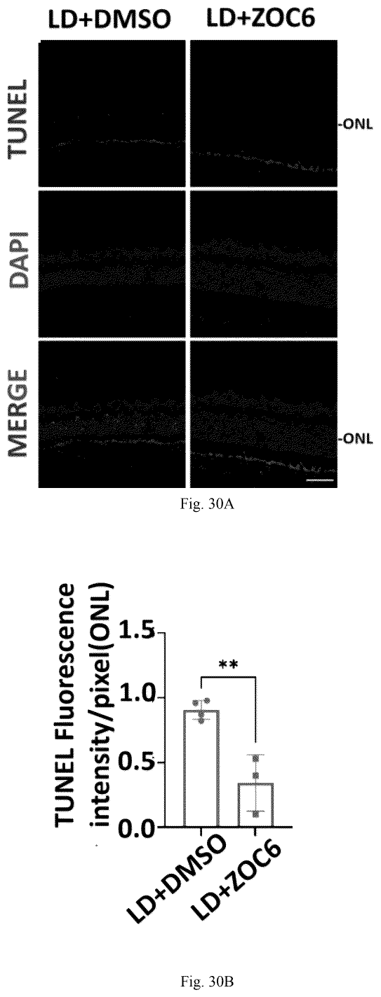

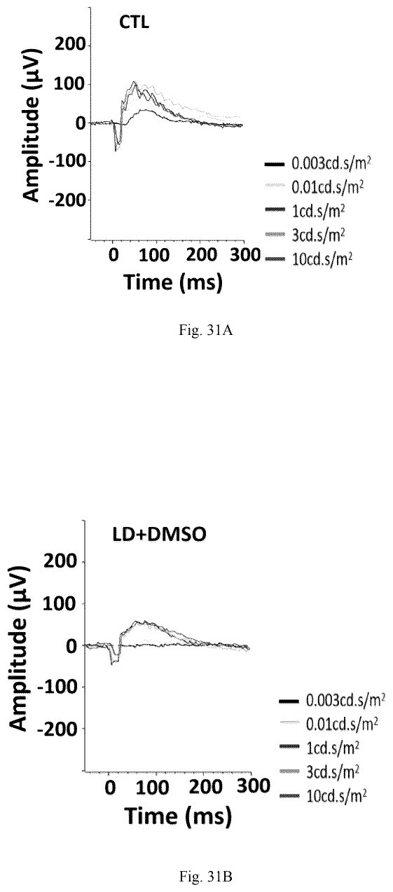

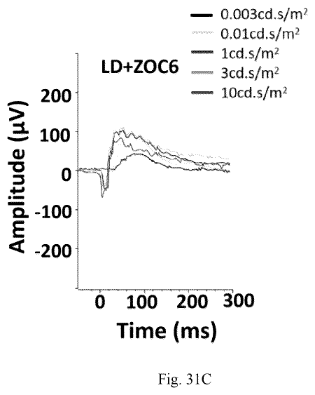

FIGS. 31A-C showed the mouse electroretinogram (ERG), which was used to evaluate the visual conduction function of mice. As shown in the figure, photodamage (LD+DMSO group) caused a decrease in a-wave and b-wave, indicating that photodamage caused photoreceptor and bipolar cell dysfunction in mice, which could be alleviated by ZOC6 (LD+ZOC6 group) treatment.

FIGS. 32A-B showed the statistical graphs of a-wave and b-wave amplitudes in the ERG of the electroretinogram of mice under different intensities of flash stimulation. It could be seen from the figure that compared with the CTL control group and the ZOC6 treatment group of mice, The amplitudes of a-wave and b-wave in the light exposure group (LD+DMSO group) mice decreased under different flash stimuli (0.003-10 cd·s/m2), while there was no significant change in the amplitudes of a-wave and b-wave in the ZOC6 treatment group (LD+ZOC6 group) compared with the control group (CTL) mice. Overall, the visual function of mice in the ZOC6 injection group was better than that in the DMSO group.

FIG. 33 showed the bipolar cell immunofluorescence staining results of PKC-α positive paraffin sections of mouse retina. PKC-α (Protein Kinase C-alpha) shown in the figure as a red signal, which served as a specific marker for bipolar cells, and its fluorescence intensity was positively correlated with cell integrity; DAPI (4′,6-diamidino-2-phenylindole) referred to a blue signal in the figure showed the distribution of retinal cell nuclei, and bipolar cells were mainly located in the inner nuclear layer (INL). MERGE (Merge Channel) means that superimpose the fluorescence signals of PKC-α (red) and DAPI (blue) to show the spatial localization of bipolar cells (red) in retinal tissue. In the electroretinogram (ERG) experiment, the decrease in b-wave amplitude caused by light exposure reflects bipolar cell dysfunction. The immunofluorescence results also showed that the synapses of bipolar cells (red) were significantly reduced after light treatment. The morphology of bipolar cells in the ZOC6 treatment group was similar to that in the control group.

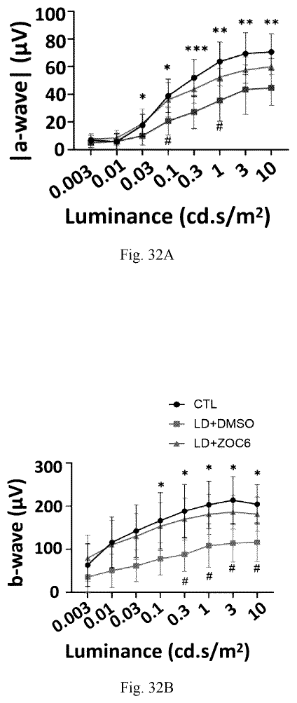

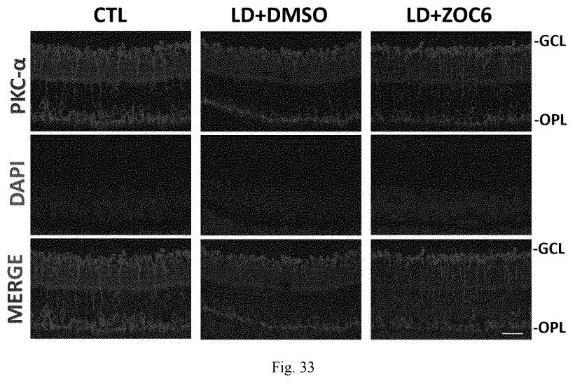

FIG. 34 showed the immunofluorescence staining results of pde6h positive photoreceptor cells in mouse retinal paraffin sections. pde6h (Phosphodiesterase 6H) is the inhibitory Y subunit of cone cell-specific cGMP phosphodiesterase (pde6h) and plays a key role in visual signal transduction. In the electroretinogram (ERG) experiment, the decrease in a-wave amplitude caused by light exposure reflected the dysfunction of photoreceptor cells. The immunofluorescence results also showed that the number of photoreceptor cells (green) significantly decreased after light treatment. The density of photoreceptor cells in the ZOC6 treatment group (LD+ZOC6 group) was similar to that in the control group.

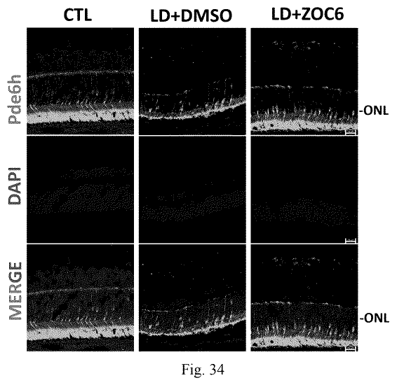

FIGS. 35A-B showed the results of GFAP immunofluorescence staining of mouse retinas (A) and the corresponding statistical analysis (B). GFAP reactive hyperplasia in the retina is one of the markers of retinal inflammatory response. As shown in the figure, compared with the control group (CTL), the GFAP signal (red) in the retina of the light group (LD+DMSO group) mice was significantly enhanced (p<0.01), while the treatment with ZOC6 (LD+ZOC6 group) could significantly inhibit the increase of GFAP signal caused by light damage (p<0.05) and suppress retinal inflammation.

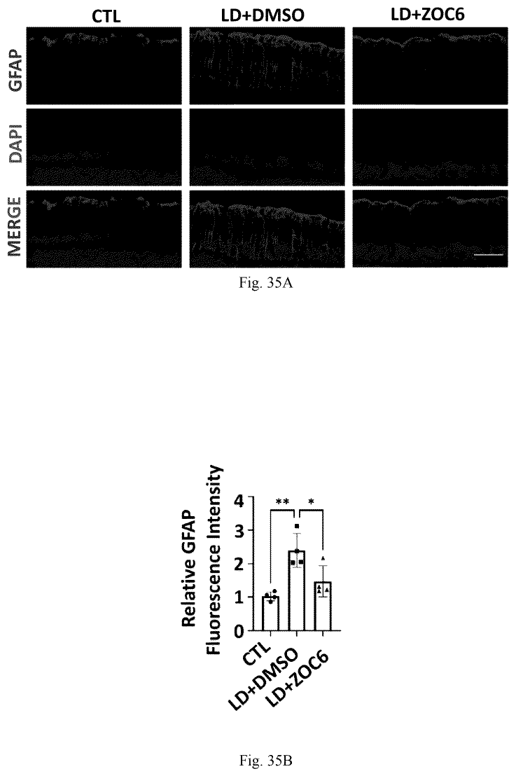

FIGS. 36A-B showed the Western Blot results of GFAP protein in the retina of mice (A) and the corresponding statistical analysis (B). It was consistent with the immunofluorescence results. As shown in the figure, the level of GFAP protein in the retina of mice in the light exposure group was significantly increased (p<0.001), while the treatment with ZOC6 could inhibit the increase in GFAP protein level caused by light damage (p<0.001) and suppress retinal inflammation.

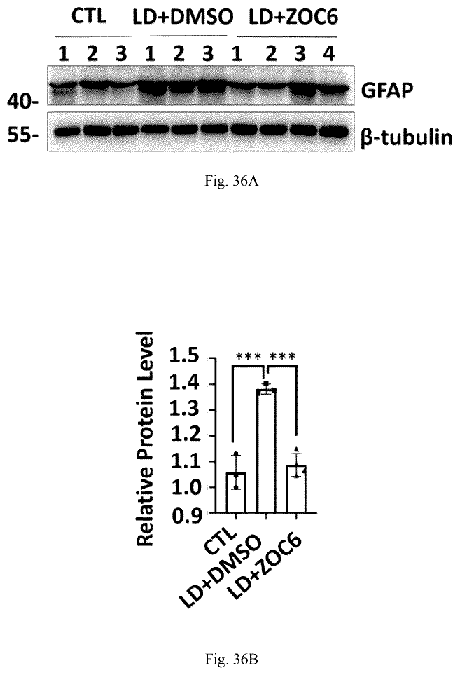

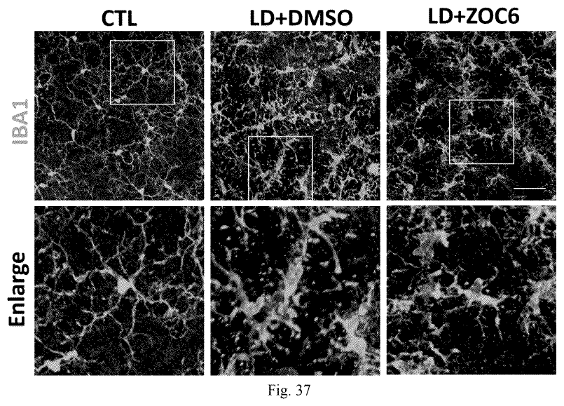

FIG. 37 showed the immunofluorescence staining results of IBA1 positive microglia in a mouse retinal mount. The increase in the number of retinal microglia as well as the morphological changes were the markers of retinal inflammatory response. As shown, IBA1 referred to microglia, and enlarge indicates the magnified picture at the box position. Compared with the control group (CTL), there were activated, amoeba-like microglia in the retina of the light-treated (LD+DMSO group) mice, and the number of IBA1 positive microglia (green) was significantly increased, while ZOC6 treatment reduced the number and activation of microglia.

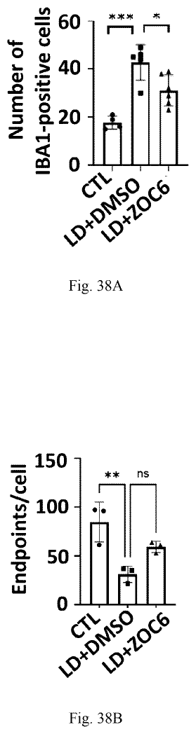

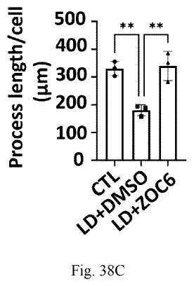

FIGS. 38A-C showed the results of morphological analysis of microglia. Light treatment significantly increased the infiltration of microglia (p<0.001), and the process length of microglia was shortened (p<0.01) and endpoints reduction (p<0.01), which were all indicators of microglia activation. Injection of ZOC6 (LD+ZOC6 group) reduced the number of microglia (p<0.05), and partially inhibited the reactive phenotype after light damage.

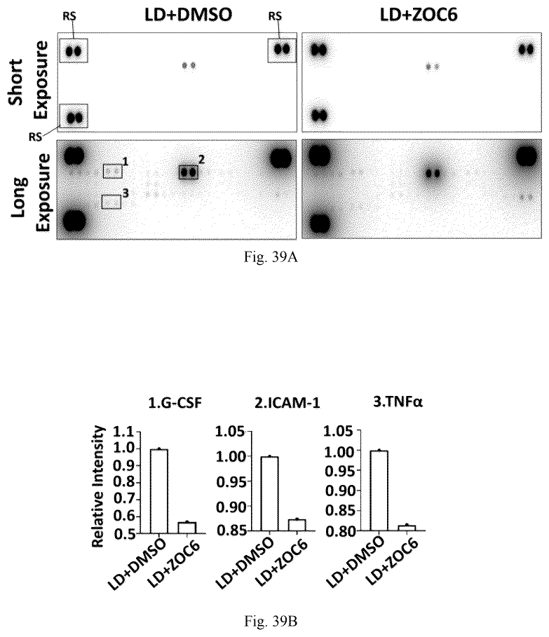

FIGS. 39A-B showed the results of cytokine immunoblotting (A) and the corresponding statistical analysis (B) for the detection of inflammatory factor expression in the mouse retina. Injection of ZOC6 (LD+ZOC6 group) inhibited the upregulation of G-CSF, ICAM-1, and TNFα after light exposure, all of which have been reported to promote retinal injury.

To sum up, these results suggested that ZOC6 could alleviate the retinal inflammatory response caused by light damage and protect the retina.

### EXPERIMENTAL EXAMPLE 11 to Degrade STING in the cGAMP-Induced Retinal Vascular Inflammation Model with ZOC6 and to Alleviate Inflammatory Responses as Well as Further Protect the Retina

In addition to the photodamage model, this present disclosure further validated the in-vivo degradation effect of ZOC6 on STING and its anti-inflammatory effect using a cGAMP-induced retinal vascular inflammation model.

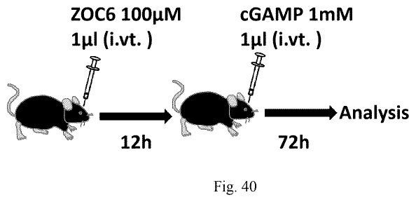

FIG. 40 is a schematic diagram of the experimental design. In this experiment, C57 mice were used. 1 μl of ZOC6 at a concentration of 100 μM was intravitreally injected. After 12 hours, 1 μl of cGAMP (Cyclic GMP-AMP) at a concentration of 1 mM was injected into the same side of the vitreous cavity. Samples were collected 3 days later for detection of relevant indicators.

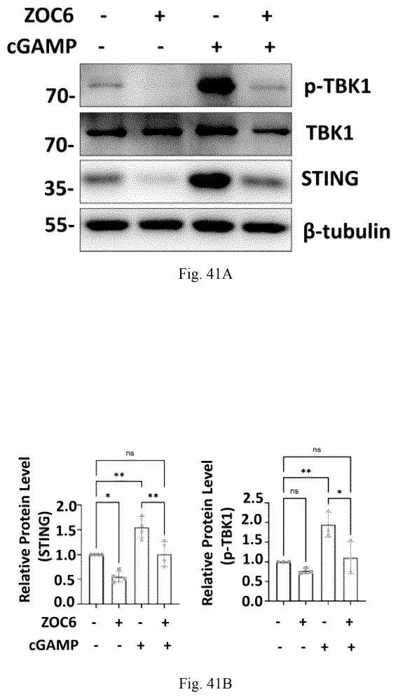

FIGS. 41A-B showed the Western Blot results (A) and the corresponding statistical analysis (B). As shown in the figure, cGAMP caused an increase in the expression level of STING in the retina (p<0.01), which in turn leaded to an increase in the phosphorylation level of downstream TBK1 (p<0.01). While the intravitreal injection of ZOC6 can degrade STING in the retina (p<0.01), and the phosphorylation level of downstream TBK1 was also inhibited (p<0.05).

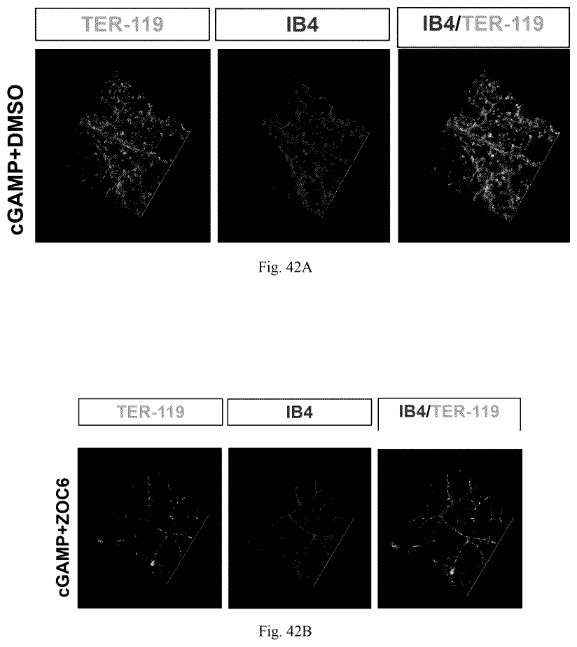

FIGS. 42A-B showed the immunofluorescence staining of retinal flat-mounted blood vessels and red blood cells. TER-119 was an antibody that specifically recognizes the surface antigen (Glycophorin A analog) of mouse red blood cells and was used to label red blood cells. IB4 (Isolectin B4) can specifically bind to glycoproteins on the surface of vascular endothelial cells, and was used to label the retinal vascular network. IB4/TER-119 merged images mean that the fluorescent signals of TER-119 (labeled with green fluorescence) and IB4 (labeled with red fluorescence) were superimposed to visually observe whether red blood cells normally exist in retinal blood vessels. As shown in the figure, cGAMP caused red blood cell leakage, while intravitreal injection of ZOC6 can significantly inhibit this leakage and protect the retina.

To sum up, the compounds A1 (ZOC6), A2 (ZOC1), A3 (ZOC2), A4 (ZOC3), A5 (ZOC4), A6 (ZOC5), B2 (ZOC8), B3 (ZOC9), and B4 (ZOC10) described in the present disclosure all had a degradation effect on STING.

SP23 is a STING degradation inhibitor on the market. The results in the prior art showed that although SP23 had good degradation efficiency, its high cytotoxicity would make it a significant limitation in subsequent applications.

The compounds in the present disclosure were developed by the applicant by altering the warhead and linker. The degradation efficiency of B2 (ZOC8) and SP23 was similar in ARPE cells. The maximum degradation rate of B2 and SP23 was 72.2% and 71.8%, respectively. In THP1 cells, compound A1 (ZOC6) and SP23 had similar degradation efficiency of STING, the maximum degradation rate of A1 was 76.6%, and the maximum degradation rate of SP23 was 87.8%.

It should be noted that, under equivalent treatment conditions, A1 (ZOC6) and B2 (ZOC8) in the compounds of the present disclosure were more than 3-7 times less cytotoxicity than SP23. Further, in-vivo experiments showed that injection of SP23 resulted in a decrease in the visual function of mice, while compound A1 (ZOC6) of the present disclosure had no effect on the visual function.

To sum up, the present disclosure provides a better option for the treatment of STING-related autoimmune and inflammatory diseases.

The embodiments disclosed above are merely illustrative of the disclosure, and are not intended to limit the present disclosure. It should be understood that any modifications, changes and replacements made by those skilled in the art without departing from the spirit of the disclosure shall fall within the scope of the disclosure defined by the appended claims.

What is claimed is:

1. A compound of formula (I), or a deuterated compound, a stereoisomer, a tautomer, a polymorph, a solvate, a N-oxide, an isotope-labeled compound, a metabolite, a prodrug or a pharmaceutically acceptable salt thereof: 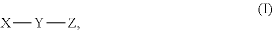
wherein X is 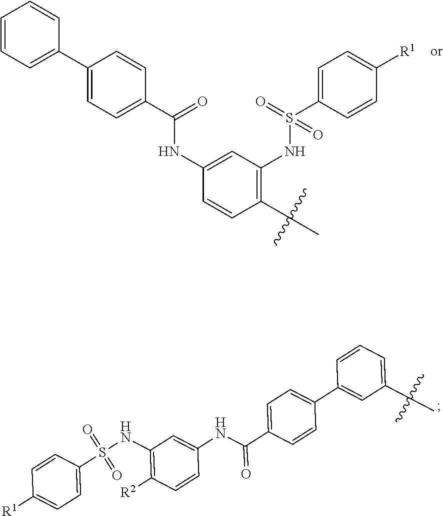
R¹ is selected from the group consisting of hydrogen, halogen, cyano, nitro, hydroxyl, —C₁₋₆ alkyl, —C₂₋₆ alkenyl, —C₂₋₆ alkynyl, halogen-substituted —C₁₋₆ alkyl, halogen-substituted —C₂₋₆ alkenyl, halogen-substituted —C₂₋₆ alkynyl, —O(C₁₋₆ alkyl), —NH₂, —NH(C₁₋₆ alkyl) and —N(C₁₋₆ alkyl)(C₁₋₆ alkyl);
R² is selected from the group consisting of hydrogen, halogen, cyano, nitro, hydroxyl, —C₁₋₆ alkyl, —C₂₋₆ alkenyl, —C₂₋₆ alkynyl, halogen-substituted —C₁₋₆ alkyl, halogen-substituted —C₂₋₆ alkenyl, halogen-substituted —C₂₋₆ alkynyl, —O(C₁₋₆ alkyl), —NH₂, —NH(C₁₋₆ alkyl) and —N(C₁₋₆ alkyl)(C₁₋₆ alkyl);
Y is a linking group; and
Z is a group capable of binding to an E3 ubiquitin ligase.

2. The compound according to claim 1, or a deuterated compound, a stereoisomer, a tautomer, a polymorph, a solvate, a N-oxide, an isotope-labeled compound, a metabolite, a prodrug or a pharmaceutically acceptable salt thereof, wherein R¹ is selected from the group consisting of hydrogen, fluorine, chlorine, bromine, iodine, cyano, hydroxyl, methyl, ethyl, n-propyl, isopropyl, vinyl, ethynyl, monofluoromethyl, difluoromethyl, trifluoromethyl, methoxy, ethoxy, —NH₂, —NH(methyl) and —N(methyl)(methyl); and
R² is selected from the group consisting of hydrogen, fluorine, chlorine, bromine, iodine, cyano, hydroxyl, methyl, ethyl, n-propyl, isopropyl, vinyl, ethynyl, monofluoromethyl, difluoromethyl, trifluoromethyl, methoxy, ethoxy, —NH₂, —NH(methyl) and —N(methyl)(methyl).

3. The compound according to claim 2, or a deuterated compound, a stereoisomer, a tautomer, a polymorph, a solvate, a N-oxide, an isotope-labeled compound, a metabolite, a prodrug or a pharmaceutically acceptable salt thereof, wherein X is

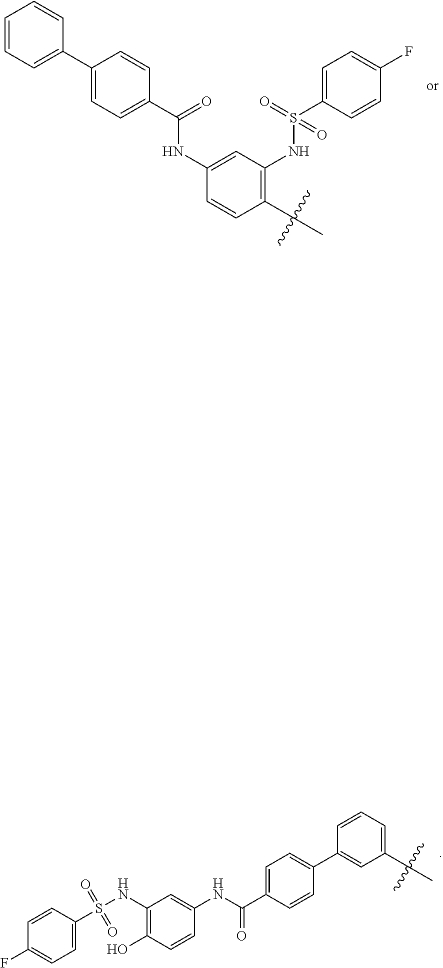 [SMILES: *c1ccc(NC(=O)c2ccc(-c3ccccc3)cc2)cc1NS(=O)(=O)c1ccc(F)cc1 ; *c1cccc(-c2ccc(C(=O)Nc3ccc(O)c(NS(=O)(=O)c4ccc(F)cc4)c3)cc2)c1]

4. The compound according to claim 1, or a deuterated compound, a stereoisomer, a tautomer, a polymorph, a solvate, a N-oxide, an isotope-labeled compound, a metabolite, a prodrug or a pharmaceutically acceptable salt thereof, wherein Y is selected from -(L^{Y})_{q}-;
q is an integer selected from 1-30; and
each L^{Y} is independently selected from the group consisting of C(R)₂, C(O), O, S, S(O), S(O)₂, NR, —CR═CR—, —C≡C—, 3-10-membered cycloalkyl, 3-10-membered heterocycloalkyl, 6-10-membered aryl, 5-10-membered heteroaryl, 5-12-membered spiro group, 5-12-membered spiroheterocyclyl, 5-12-membered bridged ring group, and 5-12-membered bridged heterocyclyl; wherein cycloalkyl, heterocycloalkyl, aryl, heteroaryl, spiro group, spiroheterocyclyl, bridged ring group and bridged heterocyclyl are unsubstituted or substituted by one, two or three R^{YL} groups;
wherein each R^{YL} group is independently selected from the group consisting of hydrogen, halogen, cyano, nitro, —C₁₋₆ alkyl, halogen-substituted —C₁₋₆ alkyl, —OR and —N(R)(R); and
each R is independently selected from the group consisting of hydrogen, halogen, —C₁₋₆ alkyl and halogen-substituted —C₁₋₆ alkyl.

5. The compound according to claim 4, or a deuterated compound, a stereoisomer, a tautomer, a polymorph, a solvate, a N-oxide, an isotope-labeled compound, a metabolite, a prodrug or a pharmaceutically acceptable salt thereof, wherein Y is 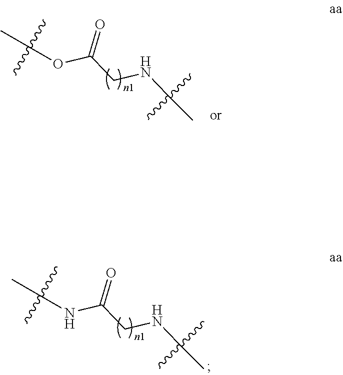
aa indicates an end linked to X;
and n1 is an integer selected from 0-10.

6. The compound according to claim 5, or a deuterated compound, a stereoisomer, a tautomer, a polymorph, a solvate, a N-oxide, an isotope-labeled compound, a metabolite, a prodrug or a pharmaceutically acceptable salt thereof, wherein Y is selected from the group consisting of: 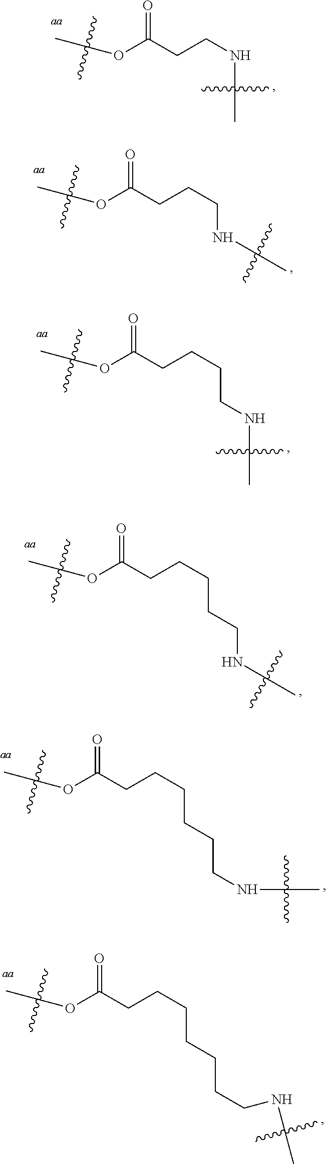 [SMILES: *NCCC(=O)O* ; *NCCCC(=O)O* ; *NCCCCC(=O)O* ; *NCCCCCC(=O)O* ; *NCCCCCCC(=O)O* ; *NCCCCCCCC(=O)O*] 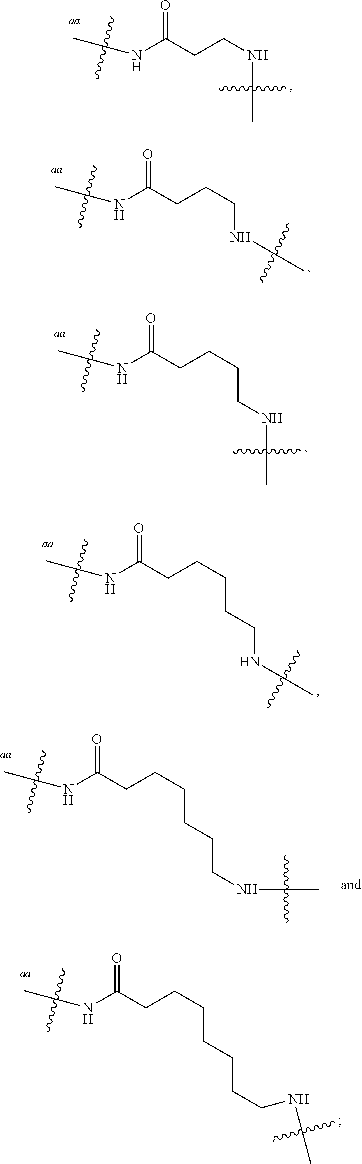 [SMILES: *NCCC(=O)N* ; *NCCCC(=O)N* ; *NCCCCC(=O)N* ; *NCCCCCC(=O)N* ; *NCCCCCCC(=O)N* ; *NCCCCCCCC(=O)N*]
wherein aa indicates an end linked to X.

7. The compound according to claim 1, or a deuterated compound, a stereoisomer, a tautomer, a polymorph, a solvate, a N-oxide, an isotope-labeled compound, a metabolite, a prodrug or a pharmaceutically acceptable salt thereof, wherein the E3 ubiquitin ligase is selected from the group consisting of Cereblon (CRBN) E3 ubiquitin ligase, von Hippel-Lindau (VHL) E3 ubiquitin ligase, X-linked inhibitor of apoptosis protein (XIAP), mouse double minute 2 homolog (MDM2) and cellular inhibitor of apoptosis protein-1 (cIAP-1).

8. The compound according to claim 1, or a deuterated compound, a stereoisomer, a tautomer, a polymorph, a solvate, a N-oxide, an isotope-labeled compound, a metabolite, a prodrug or a pharmaceutically acceptable salt thereof, wherein Z is selected from the group consisting of:

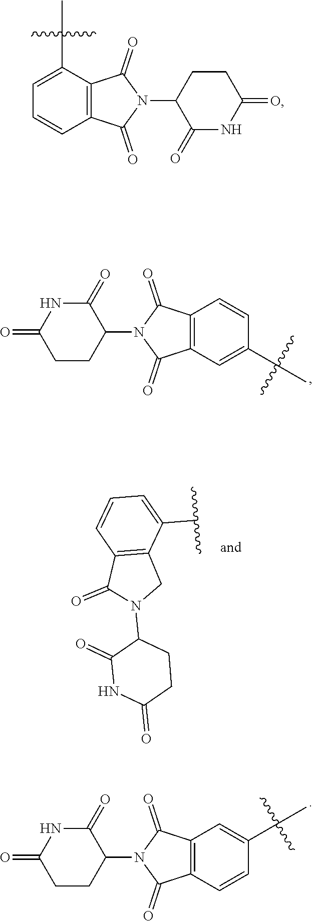 [SMILES: *c1cccc2c1C(=O)N(C1CCC(=O)NC1=O)C2=O ; *c1ccc2c(c1)C(=O)N(C1CCC(=O)NC1=O)C2=O ; *c1cccc2c1CN(C1CCC(=O)NC1=O)C2=O ; *c1ccc2c(c1)C(=O)N(C1CCC(=O)NC1=O)C2=O]

9. The compound according to claim 1, or a deuterated compound, a stereoisomer, a tautomer, a polymorph, a solvate, a N-oxide, an isotope-labeled compound, a metabolite, a prodrug or a pharmaceutically acceptable salt thereof, wherein the compound is selected from the group consisting of:

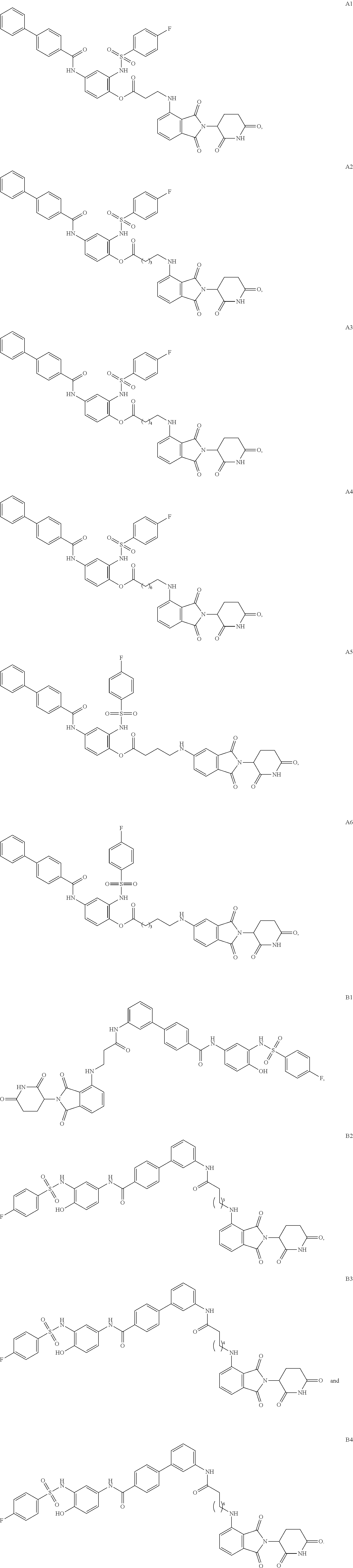

10. A pharmaceutical composition, comprising:
the compound according to claim 1, or a deuterated compound, a stereoisomer, a tautomer, a 10 polymorph, a solvate, a N-oxide, an isotope-labeled compound, a metabolite, a prodrug or a pharmaceutically acceptable salt thereof; and
a pharmaceutically acceptable carrier.

11. A method for treating a STING function-related disease in a subject in need thereof, comprising:
administering a therapeutically effective amount of the compound according to claim 1, or a deuterated compound, a stereoisomer, a tautomer, a polymorph, a solvate, a N-oxide, an isotope-labeled compound, a metabolite, a prodrug or a pharmaceutically acceptable salt thereof to the subject.

12. The method according to claim 11, wherein the STING function-related disease is selected from the group consisting of a neurodegenerative disease, an inflammatory disease, an autoimmune disease, a metabolic disease and a fibrosis disease.

13. The method according to claim 11, wherein the STING function-related disease is selected from the group consisting of STING-associated vasculopathy with onset in infancy (SAVI), Aicardi-Goutières syndrome (AGS), coatomer subunit α (COPA) syndrome caused by genetic variations in a subunit α of a coatomer protein complex, systemic lupus erythematosus (SLE), familial chilblain lupus (FCL), Parkinson's disease (PD), amyotrophic lateral sclerosis (ALS), Huntington's disease, non-alcoholic steatohepatitis (NASH), alcoholic liver disease, nerve injury, rheumatoid arthritis, renal fibrosis, systemic sclerosis, intervertebral disc degeneration, pulmonary fibrosis, aging, scleroderma, psoriasis, inflammatory bowel disease, autoimmune colitis, irritable bowel syndrome, uveitis, mucositis, diabetes and cardiovascular disease.

14. The method according to claim 11, wherein an administration route of the compound is selected from the group consisting of oral administration, intravenous injection, intramuscular injection, subcutaneous injection, transdermal administration, inhalation administration, sublingual administration, ocular administration, nasal administration, intra-articular administration and intrathecal administration.

15. A method for treating an inflammatory disease and an autoimmune disease in a subject in need thereof, comprising:
administering a therapeutically effective amount of the compound according to claim 1, or a deuterated compound, a stereoisomer, a tautomer, a polymorph, a solvate, a N-oxide, an isotope-labeled compound, a metabolite, a prodrug or a pharmaceutically acceptable salt thereof to the subject.

16. The method according to claim 15, wherein an administration route of the compound is selected from the group consisting of oral administration, intravenous injection, intramuscular injection, subcutaneous injection, transdermal administration, inhalation administration, sublingual administration, ocular administration, nasal administration, intra-articular administration and intrathecal administration.

17. The method according to claim 16, wherein the ocular administration is performed by ocular surface instillation or intravitreal injection.

18. A method for treating an ocular disease in a subject in need thereof, comprising:
administering a therapeutically effective amount of the pharmaceutical composition of claim 10 to the subjecting via intravitreal injection.
